(19) United States
(12) Patent Application Publication
(10) Pub. No.: US 2008/0292625 A1
(43) Pub. Date: Nov. 27, 2008

(54) PREVENTION AND TREATMENT OF CEREBRAL AMYLOID ANGIOPATHY

(76) Inventors: Sally Schroeter, Belmont, CA (US); Kate Dora Games, Belmont, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(21) Appl. No.: 12/106,206

(22) Filed: Apr. 18, 2008

Related U.S. Application Data
(60) Provisional application No. 60/925,228, filed on Apr. 18, 2007.

(51) Int. Cl. A61K 39/395; C07K 16/18; A61P 25/28

(52) U.S. Cl. 424/133.1; 424/172.1; 530/389.1

(57) ABSTRACT

The invention provides improved agents and methods for treatment of cerebral amyloid angiopathy (CAA) and methods to effect prophylaxis of CAA. The methods can treat CAA concurrently with Alzheimer's disease or separately. The methods can effect prophylaxis of CAA concurrently with Alzheimer's disease or separately. The methods involve administering antibody that is specific for the N-terminus of Aβ or an agent that can induce such an antibody.

### CROSS-REFERENCE TO RELATED APPLICATIONS

This application claims benefit under 35 U.S.C. §119(e) of U.S. Provisional Patent Application Ser. No. 60/925,228, filed Apr. 18, 2007, which is incorporated herein by reference in its entirety for all purposes.

### BACKGROUND OF THE INVENTION

Over expression of mutant human amyloid precursor protein (APP) in various transgenic mice leads to several Alzheimer's disease (AD)-type lesions [for reviews see D. Games et al., J Alzheimers Dis 9, 133-49 (2006); J. Gotz et al., Mol Psychiatry 9, 664-83 (2004). These include the development of parenchymal amyloid-beta (Aβ) plaques, neuritic pathology, synaptic loss, and gliosis. A number of reports have shown that active (see D. Schenk et al., Nature 400, 173-7 (1999); D. L. Dickstein et al., Faseb J 20, 426-33 (2006)) and passive (see F. Bard et al., Nat Med 6, 916-9 (2000); M. Buttini et al., J Neurosci 25, 9096-101 (2005); D. M. Wilcock et al, J Neuroinflammation 1, 24 (2004)) Aβ immunotherapeutic approaches are effective in reducing or eliminating these pathologies in preclinical studies (see R. P. Brendza & D. M. Holzman, Alzheimer Dis Assoc Disord 20, 118-23 (2006); C. A. Lernere et al., Rejuvenation Res 9, 77-84 (2006)). In addition, many studies have shown improvement in various cognitive tests (see D. M. Wilcock et al, supra; C. Janus et al., Nature 408, 979-82 (2000); D. Morgan et al., Nature 408, 982-5 (2000)). These findings are supported by mounting correlative findings from both memory testing and neuropathological examination of brains of patients who were enrolled in clinical trials of Aβ immunotherapy (AN1792), see J. A. Nicoll et al., Nat Med 9, 448-52 (2003); I. Ferrer et al., Brain Pathol 14, 11-20 (2004); S. Gilman et al., Neurology 64, 1553-62 (2005).

Recently another common aspect of AD pathology, vascular Aβ (VAβ), has been the subject of scrutiny in preclinical APP transgenic animal studies. In particular, it has been reported that passive immunization has been associated with an increase in VAβ and microhemorrhage (see D. M. Wilcock et al, supra; M. M. Racke et al., J Neurosci 25, 629-36 (2005)). However, predictive clinical implications remain unclear, especially in light of vessels in untreated and treated transgenic mice (see G. J. Burbach et al., Neurobiol Aging 28, 202-12 (2007)) and, notably, the lack of evidence for significant bleeding or stroke-related consequences in ongoing clinical trials. In addition, little is known about the degree to which VAβ is ultimately affected by Aβ immunotherapeutic approaches; for example, whether outcome measures in chronic treatment paradigms might differ from more acute studies. For instance, it is unknown whether reported increases in VAP represent a transient phenomenon associated with Aβ clearance, while longer treatment might actually prevent or reverse vascular amyloid. Finally, VAβ effects in transgenic mice may also vary according to the APP mutation employed, since the relative degree of Aβ40 versus Aβ42 production likely influences both the aggregation properties of Aβ as well as the binding efficiency of certain antibodies, particularly those with C-terminal epitopes.

### BRIEF SUMMARY OF THE INVENTION

The invention provides methods of therapeutically treating CAA. The methods comprise administering to a patient having or suspected of having CAA an effective regime of an agent. In some methods the agent is an antibody that is specific for the N-terminus of Aβ and thereby treating the patient. Optionally, the agent is an antibody that binds within residues 1-5 of Aβ. Optionally, the antibody is a humanized, human, or chimeric antibody. Optionally, the humanized antibody is 3D6. Optionally, the 3D6 humanized antibody is bapineuzumab. Optionally, the humanized antibody is 12A11.

In some methods, the agent is a fragment of Aβ. Optionally, the fragment begins at residue 1 of Aβ and ends at one of residues 5-10 of Aβ. Optionally, the fragment is Aβ 1-7. Optionally, the AD fragment is administered with a pharmaceutically acceptable adjuvant. Optionally, the Aβ fragment is linked to a carrier that helps the fragment induce antibodies to the fragment. Optionally, the carrier is linked to the C-terminus of the Aβ fragment.

Some methods of the invention further comprise determining that a patient has CAA, wherein the determining step occurs before the administration step. In some methods, the determining step determines that a patient is suffering from a clinical symptom of CAA.

In some methods of therapeutically treating CAA the patient lacks plaques characteristic of Alzheimer's disease in the brain. Optionally, the patient lacks plaques characteristic of Alzheimer's disease in the brain and the patient lacks symptoms of Alzheimer's disease. In some methods of therapeutically treating CAA, the patient has had a heart attack or stroke.

Optionally, the methods comprise administering a dosage of the antibody is between about 0.01 to about 5 mg/kg. Optionally, the methods comprise administering a dosage of the antibody between about 0.1 to about 5 mg/kg. Optionally, the methods comprise administering a dosage of about 0.5 mg/kg. Optionally, the methods comprise administering a dosage of about 1.5 mg/kg. Optionally, the methods comprise administering a dosage between about 0.5 to about 3 mg/kg. Optionally, the methods comprise administering a dosage between about 0.5 to about 1.5 mg/kg. Optionally, the methods comprise administering an antibody on multiple occasions. Optionally, the antibody is administered is weekly to quarterly. Optionally, the antibody is administered every 13 weeks. Optionally, the antibody is administered intravenously or subcutaneously.

Optionally, the antibody is administered in a regime sufficient to maintain an average serum concentration of the antibody in the patient in a range of 1-15 μg antibody/ml serum and thereby treating the patient. Optionally, the average serum concentration is within a range of 1-10 μg antibody/ml serum. Optionally, the average serum concentration is within a range of 1-5 μg antibody/ml serum. Optionally, the average serum concentration is within a range of 2-4 μg antibody/ml serum. Optionally, the antibody is administered in a regime sufficient to maintain average serum concentration of the antibody is maintained for at least one year. Optionally, the average serum concentration of the antibody is maintained for at least six months

In some methods where agent is an antibody, optionally, further comprise measuring the concentration of antibody in the serum and adjusting the regime if the measured concentration falls outside the range. In some methods where agent is an antibody, optionally, further comprise measuring the concentration of antibody in the serum and adjusting the regime if the measured concentration falls outside the range.

Optionally, the antibody is administered intravenously in a regime sufficient to maintain an average serum concentration of the antibody in the patient in a range of 1-15 μg antibody/ml serum and thereby treating the patient. Optionally, a dose of 0.5-1.0 mg/kg is administered intravenously monthly. Optionally, a dose of 0.1-1.0 mg/kg is administered intravenously monthly.

Optionally, the antibody is administered subcutaneously. Optionally, the antibody is administered subcutaneously at a frequency between weekly and monthly. Optionally, the antibody is administered subcutaneously weekly or biweekly. Optionally, the antibody is administered subcutaneously at a dose of between about 0.01 to about 0.35 mg/kg. Optionally, the antibody is administered subcutaneously at a dose of between about 0.05 to about 0.25 mg/kg. Optionally, the antibody is administered subcutaneously at a dose of between about 0.015 to about 0.2 mg/kg weekly to biweekly. Optionally, the antibody is administered subcutaneously at a dose of between about 0.05 to about 0.15 mg/kg weekly to biweekly. Optionally, the antibody is administered subcutaneously at a dose of between about 0.05 to about 0.07 mg/kg weekly. Optionally, the antibody is administered subcutaneously at a dose of 0.06 mg/kg weekly. Optionally, the antibody is administered subcutaneously at a dose of between about 0.1 to about 0.15 mg/kg biweekly.

Optionally, the antibody is administered subcutaneously at a dose of between about 0.01 to about 0.6 mg/kg and a frequency of between weekly and monthly. Optionally, the antibody is administered subcutaneously at a dose of between about 0.05 to about 0.25 mg/kg. Optionally, the antibody is administered subcutaneously at a dose of between about 0.015 to about 0.2 mg/kg weekly to biweekly. Optionally, the antibody is administered subcutaneously at a dose of between about 0.05 to about 0.15 mg/kg weekly to biweekly. Optionally, the antibody is administered subcutaneously at a dose of between about 0.05 and about 0.07 mg/kg weekly. Optionally, the antibody is administered subcutaneously at a dose of 0.06 mg/kg weekly. Optionally, the antibody is administered subcutaneously at a dose of between about 0.1 to about 0.15 mg/kg biweekly. Optionally, the antibody is administered subcutaneously at a dose of between about 0.1 to about 0.3 mg/kg monthly. Optionally, the antibody is administered at a dose of 0.2 mg/kg monthly.

Some methods of the invention further comprise monitoring for changes in signs or symptoms of CAA responsive to the administrating step. Some methods of the invention further comprise administering a second agent effective to treat CAA.

The invention provides methods of effecting prophylaxis against CAA. The methods comprise administering to a patient susceptible to CAA an effective regime of an agent. The agent is antibody that is specific for the N-terminus of Aβ or the agent induces such an antibody after administration to the patient and thereby effecting prophylaxis of the patient. The invention provides for the use of an agent, wherein the agent is an antibody that is specific for the N-terminus of Aβ or induces such an antibody after administration to the patient, in the treatment or prophylaxis of Alzheimer's disease.

The invention provides methods of reducing vascular amyloid in a patient. The methods comprise administering an antibody that is specific for the N-terminus of Aβ in a treatment regime associated with efficacious vascular amyloid removal and reduced incidence of cerebral microhemorrhage. Some methods further comprise monitoring the patient for cerebral microhemorrhage by MRI. Some methods further comprise monitoring the patient for vascular amyloid removal by PET scan. Optionally, in some methods the treatment regime is a chronic treatment regime. Optionally, in some methods the treatment regime comprises an antibody dosage between 0.01 and 5 mg/kg body weight of the patient and administered weekly to quarterly. Optionally, in some methods the dosage of the antibody is 0.1 to 5 mg/kg. Optionally, in some methods the dosage is about 0.5 mg/kg. Optionally, in some methods the dosage is about 1.5 mg/kg. Optionally, in some methods the dosage is between about 0.5 to about 3 mg/kg. Optionally, in some methods the dosage is between about 0.5 to about 1.5 mg/kg. Optionally, in some methods the dosage is administered every 13 weeks. Optionally, in some methods the antibody is administered intravenously or subcutaneously. Optionally, the agent is an antibody that binds within residues 1-5 of Aβ. Optionally, the antibody is a humanized, human, or chimeric antibody. Optionally, the humanized antibody is 3D6. Optionally, the 3D6 humanized antibody is bapineuzumab. Optionally, the humanized antibody is 12A 11.

The invention provides methods of treating Alzheimer's disease. The methods comprise administering an antibody that is specific for the N-terminus of Aβ at a dose that reduces or inhibits development of vascular amyloidogenic pathology, minimizes microhemorrhage, and or reduces or inhibits development of Aβ plaques. Optionally, in some methods the antibody binds within residues 1-5 of Aβ. Optionally, the antibody is a humanized, human, or chimeric antibody. Optionally, the humanized antibody is 3D6. Optionally, the 3D6 humanized antibody is bapineuzumab. Optionally, the humanized antibody is 12A11.

The invention provides methods of treating Alzheimer's disease that comprise administering an antibody that is specific for the N-terminus of Aβ at a dose that reduces or inhibits development of vascular amyloidogenic pathology, minimizes microhemorrhage, and or reduces or inhibits development of neuritic pathology. Optionally, in some methods the antibody binds within residues 1-5 of Aβ. Optionally, the antibody is a humanized, human, or chimeric antibody. Optionally, the humanized antibody is 3D6. Optionally, the 3D6 humanized antibody is bapineuzumab. Optionally, the humanized antibody is 12A11.

The invention provides methods for treating Alzheimer's disease that comprise administering an antibody that is specific for the N-terminus of Aβ at a dose that reduces or inhibits vascular amyloidogenic pathology, minimizes microhemorrhage, and or improves patient's cognitive function. Optionally, in some methods the antibody binds within residues 1-5 of Aβ. Optionally, the antibody is a humanized, human, or chimeric antibody. Optionally, the humanized antibody is 3D6. Optionally, the 3D6 humanized antibody is bapineuzumab. Optionally, the humanized antibody is 12A11.

Optionally, some methods of treating Alzheimer's disease the reduction or inhibition of vascular amyloidogenic pathology is a prevention of accumulation of vascular Aβ or clearance of vascular Aβ.

The invention further provides diagnostic kits suitable for use in the above methods. Such a kit comprises an antibody that specifically binds to an epitope with residues 1-10 of Aβ. Some kits bear a label describing use of the antibody for in vivo diagnosis or monitoring of Alzheimer's disease

The invention further provides kits for treatment of CAA suitable for use in the above methods. Such a kit comprises a glass vial containing a formulation. Some kits of the invention comprise a glass vial containing a formulation comprising about 0.5 to 3 mg/kg of a humanized anti-Aβ antibody. Some kits of the invention comprise a glass vial containing a formulation comprising: i. between about 10 mg to about 250 mg of a humanized anti-Aβ antibody, ii. about 4% mannitol or about 150 mM NaCl, iii. about 5 mM to about 10 mM histidine, and iv. about 10 mM methionine. Some kits contain instructions to monitor a patient to whom the formulation is administered for CAA. Optionally, the instructions comprise: i. monitoring the patient for cerebral microhemorrhage by MRI, or ii. monitoring the patient for vascular amyloid removal by PET scan.

The invention further provides kits for treatment of Alzheimer's disease suitable for use in the above methods. Such a comprises a glass vial containing a formulation comprising: i. between about 10 mg to about 250 mg of a humanized anti-Aβ antibody, ii. about 4% mannitol or about 150 mM NaCl, iii. about 5 mM to about 10 mM histidine, and iv. about 10 mM methionine. Some kits contain instructions to monitor a patient to whom the formulation is administered for Alzheimer's disease. Optionally, the instructions comprise: i. monitoring the patient for cerebral microhemorrhage by MRI, or ii. monitoring the patient for vascular amyloid removal by PET scan.

The invention further provides kits for treatment of CAA and Alzheimer's disease suitable for use in the above methods. Such a kit comprises a glass vial containing a formulation comprising: i. between about 10 mg to about 250 mg of a humanized anti-Aβ antibody, ii. about 4% mannitol or about 150 mM NaCl, iii. about 5 mM to about 10 mM histidine, and iv. about 10 mM methionine. Some kits contain instructions to monitor a patient to whom the formulation is administered for CAA and Alzheimer's disease. Optionally, the instructions comprise: i. monitoring the patient for cerebral microhemorrhage by MRI, or ii. monitoring the patient for vascular amyloid removal by PET scan.

Optionally, the antibody is administered at a dose of between about 0.05 to about 0.5 mg/kg. Optionally, the antibody is administered at a dose of between about 1 to about 40 mg and a frequency of between weekly and monthly. Optionally, the antibody is administered at a dose of between about 5 to about 25 mg and a frequency of between weekly and monthly. Optionally, the antibody is administered at a dose of between about 2.5 to about 15 mg and a frequency of between weekly and monthly.

Optionally, the antibody is administered at a dose of between about 1 to about 12 mg weekly to biweekly. Optionally, the antibody is administered at a dose of between about 2.5 to about 10 mg weekly to biweekly. Optionally, the antibody is administered at a dose of between about 2.5 to about 5 mg weekly. Optionally, the antibody is administered at a dose of between about 4 to about 5 mg weekly. Optionally, the antibody is administered at a dose of between about 7 to about 10 mg biweekly.

### BRIEF DESCRIPTION OF THE DRAWINGS

FIG. 1a shows thioflavin S staining, and FIG. 1b shows 3D6 immunolabeling of Aβ in brain midline vessels of 18-month-old PDAPP mice. FIG. 1c shows human AD tissue, and FIG. 1d shows leptomeningeal and superficial parenchymal vessels in the PDAPP mouse with VAβ immunolabeled by 3D6. Scale bars=100 μm.

FIG. 2a shows untreated 12-month-old mouse brain, FIG. 2b shows control-treated mouse brain, FIG. 2c shows 3 mg/kg 3D6-treated mouse brain, and FIG. 2d shows 3 mg/kg 266-treated mouse brain with 3D6 immunolabeling of VAβ in midline vessels. Scale bar=50 μm. Graph shows the percentage of animals in each group with none-little VAβ (white bars) and moderate VAβ (cross-hatched bars).

FIG. 3a shows control-treated brain, FIG. 3b shows 0.1 mg/kg 3D6-treated brain, FIG. 3c shows 0.3 mg/kg 3D6-treated brain, and FIG. 3d shows 3 mg/kg 3D6-treated brain with 3D6 immunolabeling of VAβ in leptomeningeal vessels. Brackets and arrows, VAβ, Scale bars=100 μm. Graph shows the percentage of animals in each group with none-little VAβ (white bars) and moderate VAβ (cross-hatched bars).

FIG. 4a shows 3D6 immunolabeling of rounded masses and bands of intact VAβ encompassing an unaffected leptomeningeal vessel in a 0.1 mg/kg 3D6-treated mouse. FIG. 4b shows 3D6 immunolabeling of patchy, eroded VAβ during partial clearance in a 0.1 mg/kg 3D6-treated mouse. Scale bar=50 μm.

FIGS. 5a and 5b show partial clearance or prevention of VAβ at lower doses of 3D6 with no evidence of microhemorrhage in most animals. FIG. 5c shows complete clearance or prevention of VAβ at 3 mg/kg 3D6 with no evidence of microhemorrhage in most animals. FIGS. 5d and 5e show microhemorrhage at sites of partial clearance at lower doses of 3D6. FIG. 5f shows microhemorrhage at sites of complete clearance at 3 mg/kg 3D6. Arrows, macrophages. Scale bar=100 μm.

FIG. 6a shows hemosiderin ratings of control and treatment groups in Study A. FIG. 6b shows hemosiderin ratings of control and treatment groups in Study B.

### DEFINITIONS

The term “substantial identity” means that two peptide sequences, when optimally aligned, such as by the programs GAP or BESTFIT using default gap weights, share at least 65 percent sequence identity, preferably at least 80 or 90 percent sequence identity, more preferably at least 95 percent sequence identity or more (e.g., 99 percent sequence identity or higher). Preferably, residue positions which are not identical differ by conservative amino acid substitutions.

For sequence comparison, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., supra). One example of algorithm that is suitable for determining percent sequence identity and sequence similarity is the BLAST algorithm, which is described in Altschul et al., J. Mol. Biol. 215:403-410 (1990). Software for performing BLAST analyses is publicly available through the National Center for Biotechnology Information (NCBI) website. Typically, default program parameters can be used to perform the sequence comparison, although customized parameters can also be used. For amino acid sequences, the BLASTP program uses as defaults a word length (W) of 3, an expectation (E) of 10, and the BLOSUM62 scoring matrix (see Henikoff & Henikoff, Proc. Natl. Acad. Sci. USA 89, 10915 (1989)).

For purposes of classifying amino acids substitutions as conservative or non-conservative, amino acids are grouped as follows: Group I (hydrophobic sidechains): norleucine, met, ala, val, leu, ile; Group II (neutral hydrophilic side chains): cys, ser, thr; Group III (acidic side chains): asp, glu; Group IV (basic side chains): asn, gln, his, lys, arg; Group V (residues influencing chain orientation): gly, pro; and Group VI (aromatic side chains): trp, tyr, phe. Conservative substitutions involve substitutions between amino acids in the same class. Non-conservative substitutions constitute exchanging a member of one of these classes for a member of another.

Therapeutic agents of the invention are typically substantially pure from undesired contaminants. This means that an agent is typically at least about 50% w/w (weight/weight) purity, as well as being substantially free from interfering proteins and contaminants. Sometimes the agents are at least about 80% w/w and, more preferably at least about 90 or about 95% w/w purity. However, using conventional protein purification techniques, homogeneous peptides of at least about 99% w/w can be obtained.

The phrase that a molecule “specifically binds” or “specifically immunoreactive” to a target refers to a binding reaction which is determinative of the presence of the molecule in the presence of a heterogeneous population of other biologics. Thus, under designated immunoassay conditions, a specified molecule binds preferentially to a particular target and does not bind in a significant amount to other biologics present in the sample. Specific binding of an antibody to a target under such conditions requires the antibody be selected for its specificity to the target. A variety of immunoassay formats may be used to select antibodies specifically immunoreactive with a particular protein. For example, solid-phase ELISA immunoassays are routinely used to select monoclonal antibodies specifically immunoreactive with a protein. See, e.g., Harlow and Lane (1988) Antibodies, A Laboratory Manual, Cold Spring Harbor Publications, New York, for a description of immunoassay formats and conditions that can be used to determine specific immunoreactivity. Specific binding between two entities means an affinity of at least 10⁶, 10⁷, 10⁸, 10⁹ M⁻¹, or 10¹⁰ M⁻¹. Affinities greater than 10⁸ M⁻¹ are preferred.

The term “antibody” or “immunoglobulin” is used to include intact antibodies and binding fragments thereof. Typically, fragments compete with the intact antibody from which they were derived for specific binding to an antigen fragment including separate heavy chains, light chains Fab, Fab′ F(ab′)2, Fabc, and Fv. Fragments are produced by recombinant DNA techniques, or by enzymatic or chemical separation of intact immunoglobulins. The term “antibody” also includes one or more immunoglobulin chains that are chemically conjugated to, or expressed as, fusion proteins with other proteins. The term “antibody” also includes bispecific antibody. A bispecific or bifunctional antibody is an artificial hybrid antibody having two different heavy/light chain pairs and two different binding sites. Bispecific antibodies can be produced by a variety of methods including fusion of hybridomas or linking of Fab′ fragments. See, e.g., Songsivilai & Lachmann, Clin. Exp. Immunol. 79:315-321 (1990); Kostelny et al., J. Immunol. 148, 1547-1553 (1992).

APP⁶⁹⁵, APP⁷⁵¹, and APP⁷⁷⁰ refer, respectively, to the 695, 751, and 770 amino acid residue long polypeptides encoded by the human APP gene. See Kang et al., Nature 325, 773 (1987); Ponte et al., Nature 331, 525 (1988); and Kitaguchi et al., Nature 331, 530 (1988). Amino acids within the human amyloid precursor protein (APP) are assigned numbers according to the sequence of the APP770 isoform.

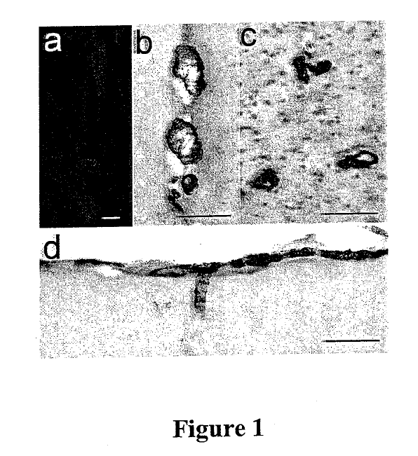

Terms such as Aβ39, Aβ40, Aβ41, Aβ42 and Aβ43 refer to an Aβ peptide containing amino acid residues 1-39, 1-40, 1-41, 1-42 and 1-43. The sequences of these peptides and their relationship to the APP precursor are illustrated by FIG. 1 of Hardy et al., TINS 20, 155-158 (1997). For example, Aβ42 has the sequence:

DAEFRHDSGYEVHHQKLVFFAEDVGSNKGAIIGLMVGGVVIAT

Aβ41, Aβ40 and Aβ39 differ from Aβ42 by the omission of Ala, Ala-Ile, and Ala-Ile-Val respectively from the C-terminal end. Aβ43 differs from Aβ42 by the presence of a Thr residue at the C-terminus.

An “antigen” is an entity to which an antibody specifically binds.

The term “epitope” or “antigenic determinant” refers to a site on an antigen to which B and/or T cells respond. B-cell epitopes can be formed both from contiguous amino acids or noncontiguous amino acids juxtaposed by tertiary folding of a protein. Epitopes formed from contiguous amino acids are typically retained on exposure to denaturing solvents whereas epitopes formed by tertiary folding are typically lost on treatment with denaturing solvents. An epitope typically includes at least 3, and more usually, at least 5 or 8-10 amino acids in a unique spatial conformation. Methods of determining spatial conformation of epitopes include, for example, x-ray crystallography and 2-dimensional nuclear magnetic resonance. See, e.g., Epitope Mapping Protocols in Methods in Molecular Biology, Vol. 66, Glenn E. Morris, Ed. (1996). Antibodies that recognize the same epitope can be identified in a simple immunoassay showing the ability of one antibody to block the binding of another antibody to a target antigen. T-cells recognize continuous epitopes of about nine amino acids for CD8 cells or about 13-15 amino acids for CD4 cells. T cells that recognize the epitope can be identified by in vitro assays that measure antigen-dependent proliferation, as determined by ³H-thymidine incorporation by primed T cells in response to an epitope (Burke et al., J. Inf. Dis. 170, 1110-19 (1994)), by antigen-dependent killing (cytotoxic T lymphocyte assay, Tigges et al., J. Immunol. 156, 3901-3910) or by cytokine secretion.

The term “immunological” or “immune” response is the development of a humoral (antibody mediated) and/or a cellular (mediated by antigen-specific T cells or their secretion products) response directed against an amyloid peptide in a recipient patient. Such a response can be an active response induced by administration of immunogen or a passive response induced by administration of antibody or primed T-cells. A cellular immune response is elicited by the presentation of polypeptide epitopes in association with Class I or Class II MHC molecules to activate antigen-specific CD4⁺ T helper cells and/or CD8⁺ cytotoxic T cells. The response may also involve activation of monocytes, macrophages, NK cells, basophils, dendritic cells, astrocytes, microglia cells, eosinophils or other components of innate immunity. The presence of a cell-mediated immunological response can be determined by proliferation assays (CD4⁺ T cells) or CTL (cytotoxic T lymphocyte) assays (see Burke, supra; Tigges, supra). The relative contributions of humoral and cellular responses to the protective or therapeutic effect of an immunogen can be distinguished by separately isolating antibodies and T-cells from an immunized syngeneic animal and measuring protective or therapeutic effect in a second subject.

An “immunogenic agent” or “immunogen” is capable of inducing an immunological response against itself on administration to a mammal, optionally in conjunction with an adjuvant.

The term “all-D” refers to peptides having ≧75%, ≧80%, ≧85%, ≧90%, ≧95%, or 100% D-configuration amino acids.

The term “naked polynucleotide” refers to a polynucleotide not complexed with colloidal materials. Naked polynucleotides are sometimes cloned in a plasmid vector.

The term “adjuvant” refers to a compound that when administered in conjunction with an antigen augments the immune response to the antigen, but when administered alone does not generate an immune response to the antigen. Adjuvants can augment an immune response by several mechanisms including lymphocyte recruitment, stimulation of B and/or T cells, and stimulation of macrophages.

The term “effective dose” or “effective dosage” is defined as an amount sufficient to achieve or at least partially achieve the desired effect. The term “therapeutically effective dose” is defined as an amount sufficient to cure or at least partially arrest the disease and its complications in a patient already suffering from the disease. Amounts effective for this use will depend upon the severity of the infection and the general state of the patient's own immune system.

Some variation of disclosed ranges and is permissible as for example due to errors in measurement. Such variation is designated by the term “about” in reference to ranges or doses.

The term “patient” includes human and other mammalian subjects that receive either prophylactic or therapeutic treatment.

Competition between antibodies is determined by an assay in which the immunoglobulin under test inhibits specific binding of a reference antibody to a common antigen, such as Aβ. Numerous types of competitive binding assays are known, for example: solid phase direct or indirect radioimmunoassay (RIA), solid phase direct or indirect enzyme immunoassay (EIA), sandwich competition assay (see Stahli et al., Methods in Enzymology 9:242-253 (1983)); solid phase direct biotin-avidin EIA (see Kirkland et al., J. Immunol. 137:3614-3619 (1986)); solid phase direct labeled assay, solid phase direct labeled sandwich assay (see Harlow and Lane, Antibodies, A Laboratory Manual, Cold Spring Harbor Press (1988)); solid phase direct label RIA using I-125 label (see Morel et al., Molec. Immunol. 25(1):7-15 (1988)); solid phase direct biotin-avidin EIA (Cheung et al., Virology 176:546-552 (1990)); and direct labeled RIA (Moldenhauer et al., Scand. J. Immunol. 32:77-82 (1990)). Typically, such an assay involves the use of purified antigen bound to a solid surface or cells bearing either of these, an unlabelled test immunoglobulin and a labeled reference immunoglobulin. Competitive inhibition is measured by determining the amount of label bound to the solid surface or cells in the presence of the test immunoglobulin. Usually the test immunoglobulin is present in excess. Antibodies identified by competition assay (competing antibodies) include antibodies binding to the same epitope as the reference antibody and antibodies binding to an adjacent epitope sufficiently proximal to the epitope bound by the reference antibody for steric hindrance to occur. Usually, when a competing antibody is present in excess, it will inhibit specific binding of a reference antibody to a common antigen by at least 50 or 75%.

The term “symptom” or “clinical symptom” refers to a subjective evidence of a disease, such as altered gait, as perceived by the patient. A “sign” refers to objective evidence of a disease as observed by a physician.

Compositions or methods “comprising” one or more recited elements may include other elements not specifically recited.

### DETAILED DESCRIPTION OF THE INVENTION

### I. General

The invention provides methods of effecting prophylaxis and treatment of cerebral amyloid angiopathy (CAA), a disease characterized by presence of vascular deposits of Aβ peptide. These vascular deposits are distinct from the parenchyal deposits that are the hallmark of Alzheimer's disease. Most Alzheimer's patients are affected by at least mild CAA. However, CAA can also occur independent of symptoms and/or characteristic pathology of Alzheimer's disease. CAA is also associated with symptoms not generally associated with Alzheimer's disease, such as strokes. The invention provides methods of effecting prophylaxis or treating CAA whether it occurs alone or concurrently with Alzheimer's disease. In patients having concurrent Alzheimer's disease and CAA, the methods can treat both diseases simultaneously. In patients having neither disease, the methods can effect prophylaxis against both diseases. In patients having CAA but not Alzheimer's disease, the methods can treat CAA and effect prophylaxis of Alzheimer's disease. The methods involve active or passive immunotherapy. In passive immunotherapy, an antibody binding to an epitope within residues 1-10 of Aβ is administered. In active immunotherapy, an agent is administered, such as an Aβ fragment that can induce such an antibody. Although an understanding of mechanism is not essential for practice of the invention, it is believed that the antibodies bind to vascular deposits of Aβ and thereby promotes clearing of the deposits.

### II. Agents

The present methods employ an agent that either is an antibody to the N-terminus of Aβ (passive administration) or is capable of inducing such an antibody on administration to a patient. Such agents have been previously described in the scientific and patent literature in connection with immunotherapy of Alzheimer's disease (see WO 98/25386 and WO 00/72880).

A. Active Immunotherapy

Aβ, also known as β-amyloid peptide, or A4 peptide (see U.S. Pat. No. 4,666,829; Glenner & Wong, Biochem. Biophys. Res. Commun. 120, 1131 (1984)), is a peptide of 39-43 amino acids, which is the principal component of characteristic plaques of Alzheimer's disease. Aβ is generated by processing of a larger protein APP by two enzymes, termed β and γ secretases (see Hardy, TINS 20, 154 (1997)). Known mutations in APP associated with Alzheimer's disease occur proximate to the site of β or γ secretase, or within Aβ. For example, position 717 is proximate to the site of γ-secretase cleavage of APP in its processing to Aβ, and positions 670/671 are proximate to the site of β-secretase cleavage. It is believed that the mutations cause AD by interacting with the cleavage reactions by which Aβ is formed so as to increase the amount of the 42/43 amino acid form of Aβ generated.

Aβ has the unusual property that it can fix and activate both classic and alternate complement cascades. In particular, it binds to Clq and ultimately to C3bi. This association facilitates binding to macrophages leading to activation of B cells. In addition, C3bi breaks down further and then binds to CR2 on B cells in a T cell dependent manner leading to a 10,000 fold increase in activation of these cells. This mechanism causes Aβ to generate an immune response in excess of that of other antigens.

Preferred agents for active administration are fragments beginning at residue 1 of Aβ and ending between one of residues 5-10. Such fragments when linked to an appropriate carrier are capable or inducing antibodies that specifically bind to the N-terminus of Aβ. Such fragments are lacking naturally occurring self T-cell epitopes that have been associated with undesired side effects in clinical trials of intact Aβ. Preferred immunogenic fragments include Aβ1-5, 1-6, and 1-7, 1-10, 3-7, 1-3, and 1-4. The designation Aβ1-5 for example, indicates a fragment including residues 1-5 of Aβ and lacking other residues of Aβ.

Aβ-derived diffusible ligands (ADDLs), ADDL-surrogates, ADDL-binding molecules can also be used for active immunotherapy. See e.g., WO 2004/031400, incorporated by reference in its entirety for all purposes.

Optionally, fragments of Aβ are conjugated to carrier to help induce antibodies to the fragment. Some agents for inducing an immune response contain the appropriate epitope for inducing an immune response against amyloid but are too small to be immunogenic. In this situation, a peptide immunogen can be linked to a suitable carrier molecule to form a conjugate which helps elicit an immune response. Suitable carriers include serum albumins, keyhole limpet hemocyanin, immunoglobulin molecules, thyroglobulin, ovalbumin, tetanus toxoid, or a toxoid from other pathogenic bacteria, such as diphtheria (for example, CRM197), E. coli, cholera, or H. pylori, or an attenuated toxin derivative. T cell epitopes are also suitable carrier molecules. Some conjugates can be formed by linking agents of the invention to an immunostimulatory polymer molecule (e.g., tripalmitoyl-5-glycerine cysteine (Pam₃Cys), mannan (a manose polymer), or glucan (a beta 1→2 polymer)), cytokines (e.g., IL-1, IL-1 alpha and beta peptides, IL-2, gamma-INF, IL-1 0, GM-CSF), and chemokines (e.g., MIP1 alpha and beta, and RANTES). Immunogenic agents can also be linked to peptides that enhance transport across tissues, as described in O'Mahony, WO 97/17613 and WO 97/17614. Immunogens may be linked to the carries with or with out spacer amino acids (e.g., gly-gly).

Some conjugates can be formed by linking agents of the invention to at least one T cell epitope. Some T cell epitopes are promiscuous while other T cell epitopes are universal. Promiscuous T cell epitopes are capable of enhancing the induction of T cell immunity in a wide variety of subjects displaying various HLA types. In contrast to promiscuous T cell epitopes, universal T cell epitopes are capable of enhancing the induction of T cell immunity in a large percentage, e.g., at least 75%, of subjects displaying various HLA molecules encoded by different HLA-DR alleles.

A large number of naturally occurring T-cell epitopes exist, such as, tetanus toxoid (e.g., the P2 and P30 epitopes), hepatitis B surface antigen, pertussis, toxoid, measles virus F protein, Chlamydia trachomitis major outer membrane protein, diphtheria toxoid, Plasmodium falciparum circumsporozite T, Plasmodium falciparum CS antigen, Schistosoma mansoni triose phosphate isomersae, Escherichia coli TraT, and influenza virus hemagluttinin (HA). The immunogenic peptides of the invention can also be conjugated to the T-cell epitopes described in Sinigaglia F. et al., Nature, 336:778-780 (1988); Chicz R. M. et al., J. Exp. Med., 178:27-47 (1993); Hammer J. et al., Cell 74:197-203 (1993); Falk K. et al., Immunogenetics, 39:230-242 (1994); WO 98/23635; and, Southwood S. et al. J. Immunology, 160:3363-3373 (1998) (each of which is incorporated herein by reference for all purposes). Further examples include:

Influenza hemaglutinin: HA₃₀₇₋₃₁₉
Malaria CS: T3 epitope EKKIAKMEKASSVFNV
Hepatitis B surface antigen: HBsAg₁₉₋₂₈ FFLLTRILTI
Heat shock protein 65: hsp65₁₅₃₋₁₇₁ DQSIGDLIAEAMDKVGNEG

### Bacille Calmette-Guerin: QVHFQPLPPAVVKL

Tetanus toxoid: TT₈₃₀₋₈₄₄ QYIKANSKFIGITEL
Tetanus toxoid: TT₉₄₇₋₉₆₇ FNNFTVSFWLRVPKVSASHLE
HIV gp120 Ti: KQIINMWQEVGKAMYA

Some examples of conjugates include:

AN90549 (Aβ1-7-Tetanus toxoid 830-844 in a MAP4 configuration):
DAEFRHD-QYIKANSKFIGITEL
   AN90550 (Aβ 1-7-Tetanus toxoid 947-967 in a MAP4 configuration):
DAEFRHD-FNNFTVSFWLRVPKVSASHLE
   AN90542 (Aβ 1-7-Tetanus toxoid 830-844+947-967 in a linear configuration):
DAEFRHD-QYIKANSKFIGITELFNNFTVSFWLRVPKVSASHLE

PADRE peptide (all in linear configurations), wherein X is preferably cyclohexylalanine, tyrosine or phenylalanine, with cyclohexylalanine being most preferred:

### AN90562 (PADRE-Aβ1-7):

AKXVAAWTLAAA-DAEFRHD

### AN90543 (3 PADRE-Aβ1-7):

DAEFRHD-DAEFRHD-DAEFRHD-AKXVAAWTLKAAA

Other examples of fusion proteins (immunogenic epitope of Aβ bolded) include:

### AKXVAAWTLKAAA-DAEFRHD-DAEFRHD-DAEFRHD

### DAEFRHD-AKXVAAWTLKAAA

### DAEFRHD-ISQAVHAAHAEINEAGR

### FRHDSGY-ISQAVHAAHAEINEAGR

### EFRHDSG-ISQAVHAAHAEINEAGR

### PKYVKQNTLKLAT-DAEFRHD-DAEFRHD-DAEFRHD

### DAEFRHD-PKYVKQNTLKLAT-DAEFRHD

### DAEFRHD-DAEFRHD-DAEFRHD-PKYVKQNTLKLAT

### DAEFRHD-DAEFRHD-PKYVKQNTLKLAT

### DAEFRHD-PKYVKQNTLKLAT-EKKIAKMEKAS SVFNV-QYIKANSKFIGITEL-FNNFTVSFWLRVPKVSASHLE-DAEFRHD

### DAEFRHD-DAEFRHD-DAEFRHD-QYIKANSKFIGITELNNFTVSFWLRVPKVSASHLE

### DAEFRHD-QYIKANSKFIGITELCFNNFTVSFWLRVPKVSASHLE

### DAEFRHD-QYIKANSKFIGITELCFNNFTVSFWLRVPKVSASHLE-DAEFRHD

DAEFRHD-QYIKANSKFIGITEL on a 2 branched resin.

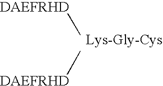

Fragments of Aβ such as Aβ1-6 are conjugated to carriers, such as virus-like-particles (VLPs) and subunits of VLPs, to help induce antibodies to the fragment. See e.g., WO 2004/016282 and US 20040141984, each of which is incorporated by reference in its entirety for all purposes.

### B. Passive Immunotherapy

Passive immunotherapy is effected using an antibody that is specific for the N-terminus Aβ. An “N-terminal epitope”, is an epitope or antigenic determinant located within or including the N-terminus of the Aβ peptide. Exemplary N-terminal epitopes include residues within amino acids 1-10 or 1-12 of Aβ, preferably from residues 1-3, 1-4, 1-5, 1-6, 1-7, 2-6, 2-7, 3-6, or 3-7 of Aβ. Other exemplary N-terminal epitopes start at residues 1-3 and end at residues 7-11 of Aβ. Additional exemplary N-terminal epitopes include residues 2-4, 2-5, 2-6, 2-7 or 2-8 of Aβ, residues 3-5, 3-6, 3-7, 3-8 or 3-9 of Aβ, or residues 4-7, 4-8, 4-9 or 4-10 of Aβ.

When an antibody is said to bind to an epitope within specified residues, such as Aβ3-7, what is meant is that the antibody specifically binds to a polypeptide containing the specified residues (i.e., Aβ3-7 in this an example). Such an antibody does not necessarily contact every residue within Aβ3-7. Nor does every single amino acid substitution or deletion within Aβ3-7 necessarily significantly affect binding affinity. In various embodiments, an Aβ antibody is end-specific. As used herein, the term “end-specific” refers to an antibody that specifically binds to the N-terminal or C-terminal residues of an Aβ peptide but that does not recognize the same residues when present in a longer Aβ species comprising the residues or in APP. Preferred antibodies have human IgG1 isotype.

Preferred anti Aβ antibodies for passive immunotherapy include a humanized anti-Aβ antibody, for example, a humanized 3D6 antibody, a humanized 12B4 antibody, or a humanized 12A11 antibody.

Antibodies for passive immunotherapy may be provided by a variety of techniques including those described in US 20040038304, US 20070020685, US 20060257396, US 20060160184, US 20060134098, US 20050255552, US 20050008625, US 20040132066, US 20040038317, US 20030198971, and US 20030157579 all of which are incorporated by reference in their entirety herein for all purposes.

Antibodies

i. General Characteristics of Immunoglobulins

The basic antibody structural unit is known to comprise a tetramer of subunits. Each tetramer is composed of two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxy-terminal portion of each chain defines a constant region primarily responsible for effector function.

Light chains are classified as either kappa or lambda. Heavy chains are classified as gamma, mu, alpha, delta, or epsilon, and define the antibody's isotype as IgG, IgM, IgA, IgD and IgE, respectively. Within light and heavy chains, the variable and constant regions are joined by a “J” region of about 12 or more amino acids, with the heavy chain also including a “D” region of about 10 more amino acids. (See generally, Fundamental Immunology, Ch. 7 (W. Paul, ed., Raven Press, N.Y., 2nd. ed. 1989), incorporated by reference in its entirety for all purposes).

The variable regions of each light/heavy chain pair form the antibody binding site. Thus, an intact antibody has two binding sites. Except in bifunctional or bispecific antibodies, the two binding sites are the same. The chains all exhibit the same general structure of relatively conserved framework regions (FR) joined by three hypervariable regions, also called complementarity determining regions or CDRs. The CDRs from the two chains of each pair are aligned by the framework regions, enabling binding to a specific epitope. From N-terminal to C-terminal, both light and heavy chains comprise the domains FR1, CDR1, FR2, CDR2, FR3, CDR3 and FR4. The assignment of amino acids to each domain is in accordance with the definitions of Kabat, Sequences of Proteins of Immunological Interest (National Institutes of Health, Bethesda, Md., 1987 and 1991); Chothia & Lesk, J. Mol. Biol. 196:901-917 (1987); or Chothia et al., Nature 342:878-883 (1989).

ii. Production of Nonhuman Antibodies

The production of non-human monoclonal antibodies, e.g., murine, guinea pig, primate, rabbit or rat, can be accomplished by, for example, immunizing the animal with Aβ. A longer polypeptide comprising Aβ or an immunogenic fragment of Aβ or anti-idiotypic antibodies to an antibody to Aβ can also be used. See Harlow & Lane, Antibodies, A Laboratory Manual (CSHP NY, 1988) (incorporated by reference for all purposes). Such an immunogen can be obtained from a natural source, by peptide synthesis, or by recombinant expression. Optionally, the immunogen can be administered fused or otherwise complexed with a carrier protein, as described below. Optionally, the immunogen can be administered with an adjuvant. Several types of adjuvant can be used as described below. Complete Freund's adjuvant followed by incomplete adjuvant is preferred for immunization of laboratory animals. Rabbits or guinea pigs are typically used for making polyclonal antibodies. Mice are typically used for making monoclonal antibodies. Antibodies are screened for specific binding to Aβ. Optionally, antibodies are further screened for binding to a specific region of Aβ. The latter screening can be accomplished by determining binding of an antibody to a collection of deletion mutants of an Aβ peptide and determining which deletion mutants bind to the antibody. Binding can be assessed, for example, by Western blot or ELISA. The smallest fragment to show specific binding to the antibody defines the epitope of the antibody. Alternatively, epitope specificity can be determined by a competition assay is which a test and reference antibody compete for binding to Aβ. If the test and reference antibodies compete, then they bind to the same epitope or epitopes sufficiently proximal that binding of one antibody interferes with binding of the other. The preferred isotype for such antibodies is mouse isotype IgG2a or equivalent isotype in other species. Mouse isotype IgG2a is the equivalent of human isotype IgG1.

iii. Chimeric and Humanized Antibodies

Chimeric and humanized antibodies have the same or similar binding specificity and affinity as a mouse or other nonhuman antibody that provides the starting material for construction of a chimeric or humanized antibody. Chimeric antibodies are antibodies whose light and heavy chain genes have been constructed, typically by genetic engineering, from immunoglobulin gene segments belonging to different species. For example, the variable (V) segments of the genes from a mouse monoclonal antibody may be joined to human constant (C) segments, such as IgG1 and IgG4. Human isotype IgG1 is preferred. In some methods, the isotype of the antibody is human IgG1. IgM antibodies can also be used in some methods. A typical chimeric antibody is thus a hybrid protein consisting of the V or antigen-binding domain from a mouse antibody and the C or effector domain from a human antibody.

Humanized antibodies have variable region framework residues substantially from a human antibody (termed an acceptor antibody) and complementarity determining regions substantially from a mouse-antibody, (referred to as the donor immunoglobulin). See, Queen et al., Proc. Natl. Acad. Sci. USA 86:10029-10033 (1989), WO 90/07861, U.S. Pat. No. 5,693,762, U.S. Pat. No. 5,693,761, U.S. Pat. No. 5,585,089, U.S. Pat. No. 5,530,101, and Winter, U.S. Pat. No. 5,225,539 (each of which is incorporated by reference in its entirety for all purposes). The constant region(s), if present, are also substantially or entirely from a human immunoglobulin. The human variable domains are usually chosen from human antibodies whose framework sequences exhibit a high degree of sequence identity with the murine variable region domains from which the CDRs were derived. The heavy and light chain variable region framework residues can be derived from the same or different human antibody sequences. The human antibody sequences can be the sequences of naturally occurring human antibodies or can be consensus sequences of several human antibodies. See Carter et al., WO 92/22653. Certain amino acids from the human variable region framework residues are selected for substitution based on their possible influence on CDR conformation and/or binding to antigen. Investigation of such possible influences is by modeling, examination of the characteristics of the amino acids at particular locations, or empirical observation of the effects of substitution or mutagenesis of particular amino acids.

For example, when an amino acid differs between a murine variable region framework residue and a selected human variable region framework residue, the human framework amino acid should usually be substituted by the equivalent framework amino acid from the mouse antibody when it is reasonably expected that the amino acid:

(1) noncovalently binds antigen directly,
(2) is adjacent to a CDR region,
(3) otherwise interacts with a CDR region (e.g. is within about 6 A of a CDR region), or
(4) participates in the VL-VH interface.

Other candidates for substitution are acceptor human framework amino acids that are unusual for a human immunoglobulin at that position. These amino acids can be substituted with amino acids from the equivalent position of the mouse donor antibody or from the equivalent positions of more typical human immunoglobulins. Other candidates for substitution are acceptor human framework amino acids that are unusual for a human immunoglobulin at that position. The variable region frameworks of humanized immunoglobulins usually show at least 85% sequence identity to a human variable region framework sequence or consensus of such sequences.

iv. Human Antibodies

Human antibodies against Aβ are provided by a variety of techniques described below. Some human antibodies are selected by competitive binding experiments, or otherwise, to have the same epitope specificity as a particular mouse antibody, such as one of the mouse monoclonals described in Example XI. Human antibodies can also be screened for a particular epitope specificity by using only a fragment of Aβ as the immunogen, and/or by screening antibodies against a collection of deletion mutants of Aβ. Human antibodies preferably have isotype specificity human IgG1.

(1) Trioma Methodology

The basic approach and an exemplary cell fusion partner, SPAZ-4, for use in this approach have been described by Oestberg et al., Hybridoma 2:361-367 (1983); Oestberg, U.S. Pat. No. 4,634,664; and Engleman et al., U.S. Pat. No. 4,634,666 (each of which is incorporated by reference in its entirety for all purposes). The antibody-producing cell lines obtained by this method are called triomas, because they are descended from three cells-two human and one mouse. Initially, a mouse myeloma line is fused with a human B-lymphocyte to obtain a non-antibody-producing xenogeneic hybrid cell, such as the SPAZ-4 cell line described by Oestberg, supra. The xenogeneic cell is then fused with an immunized human B-lymphocyte to obtain an antibody-producing trioma cell line. Triomas have been found to produce antibody more stably than ordinary hybridomas made from human cells.

The immunized B-lymphocytes are obtained from the blood, spleen, lymph nodes or bone marrow of a human donor. If antibodies against a specific antigen or epitope are desired, it is preferable to use that antigen or epitope thereof for immunization. Immunization can be either in vivo or in vitro. For in vivo immunization, B cells are typically isolated from a human immunized with Aβ, a fragment thereof, larger polypeptide containing Aβ or fragment, or an anti-idiotypic antibody to an antibody to Aβ. In some methods, B cells are isolated from the same patient who is ultimately to be administered antibody therapy. For in vitro immunization, B-lymphocytes are typically exposed to antigen for a period of 7-14 days in a media such as RPMI-1640 (see Engleman, supra) supplemented with 10% human plasma.

The immunized B-lymphocytes are fused to a xenogeneic hybrid cell such as SPAZ-4 by well-known methods. For example, the cells are treated with 40-50% polyethylene glycol of MW 1000-4000, at about 37 degrees C., for about 5-10 min. Cells are separated from the fusion mixture and propagated in media selective for the desired hybrids (e.g., HAT or AH). Clones secreting antibodies having the required binding specificity are identified by assaying the trioma culture medium for the ability to bind to Aβ or a fragment thereof. Triomas producing human antibodies having the desired specificity are subcloned by the limiting dilution technique and grown in vitro in culture medium. The trioma cell lines obtained are then tested for the ability to bind Aβ or a fragment thereof.

Although triomas are genetically stable they do not produce antibodies at very high levels. Expression levels can be increased by cloning antibody genes from the trioma into one or more expression vectors, and transforming the vector into standard mammalian, bacterial or yeast cell lines.

(2) Transgenic Non-Human Mammals

Human antibodies against Aβ can also be produced from non-human transgenic mammals having transgenes encoding at least a segment of the human immunoglobulin locus. Usually, the endogenous immunoglobulin locus of such transgenic mammals is functionally inactivated. Preferably, the segment of the human immunoglobulin locus includes unrearranged sequences of heavy and light chain components. Both inactivation of endogenous immunoglobulin genes and introduction of exogenous immunoglobulin genes can be achieved by targeted homologous recombination, or by introduction of YAC chromosomes. The transgenic mammals resulting from this process are capable of functionally rearranging the immunoglobulin component sequences, and expressing a repertoire of antibodies of various isotypes encoded by human immunoglobulin genes, without expressing endogenous immunoglobulin genes. The production and properties of mammals having these properties are described in detail by, e.g., Lonberg et al., WO93/1222, U.S. Pat. No. 5,877,397, U.S. Pat. No. 5,874,299, U.S. Pat. No. 5,814,318, U.S. Pat. No. 5,789,650, U.S. Pat. No. 5,770,429, U.S. Pat. No. 5,661,016, U.S. Pat. No. 5,633,425, U.S. Pat. No. 5,625,126, U.S. Pat. No. 5,569,825, U.S. Pat. No. 5,545,806, Nature 148, 1547-1553 (1994), Nature Biotechnology 14, 826 (1996), Kucherlapati, WO 91/10741 (each of which is incorporated by reference in its entirety for all purposes). Transgenic mice are particularly suitable. Anti-Aβ antibodies are obtained by immunizing a transgenic nonhuman mammal, such as described by Lonberg or Kucherlapati, supra, with Aβ or a fragment thereof. Monoclonal antibodies are prepared by, e.g., fusing B-cells from such mammals to suitable myeloma cell lines using conventional Kohler-Milstein technology. Human polyclonal antibodies can also be provided in the form of serum from humans immunized with an immunogenic agent. Optionally, such polyclonal antibodies can be concentrated by affinity purification using Aβ or other amyloid peptide as an affinity reagent.

(3) Phage Display Methods

A further approach for obtaining human anti-Aβ antibodies is to screen a DNA library from human B cells according to the general protocol outlined by Huse et al., Science 246:1275-1281 (1989). As described for trioma methodology, such B cells can be obtained from a human immunized with Aβ fragments, longer polypeptides containing Aβ or fragments, or anti-idiotypic antibodies. Optionally, such B cells are obtained from a patient who is ultimately to receive antibody treatment. Antibodies binding to Aβ or a fragment thereof are selected. Sequences encoding such antibodies (or binding fragments) are then cloned and amplified. The protocol described by Huse is rendered more efficient in combination with phage-display technology. See, e.g., Dower et al., WO 91/17271 and McCafferty et al., WO 92/01047, U.S. Pat. No. 5,877,218, U.S. Pat. No. 5,871,907, U.S. Pat. No. 5,858,657, U.S. Pat. No. 5,837,242, U.S. Pat. No. 5,733,743 and U.S. Pat. No. 5,565,332 (each of which is incorporated by reference in its entirety for all purposes). In these methods, libraries of phage are produced in which members display different antibodies on their outer surfaces. Antibodies are usually displayed as Fv or Fab fragments. Phage displaying antibodies with a desired specificity are selected by affinity enrichment to an Aβ peptide or fragment thereof.

In a variation of the phage-display method, human antibodies having the binding specificity of a selected murine antibody can be produced. See Winter, WO 92/20791. In this method, either the heavy or light chain variable region of the selected murine antibody is used as a starting material. If, for example, a light chain variable region is selected as the starting material, a phage library is constructed in which members display the same light chain variable region (i.e., the murine starting material) and a different heavy chain variable region. The heavy chain variable regions are obtained from a library of rearranged human heavy chain variable regions. A phage showing strong specific binding for Aβ (e.g., at least 10⁸ and preferably at least 10⁹ M⁻¹) is selected. The human heavy chain variable region from this phage then serves as a starting material for constructing a further phage library. In this library, each phage displays the same heavy chain variable region (i.e., the region identified from the first display library) and a different light chain variable region. The light chain variable regions are obtained from a library of rearranged human variable light chain regions. Again, phage showing strong specific binding for Aβ are selected. These phage display the variable regions of completely human anti-Aβ antibodies. These antibodies usually have the same or similar epitope specificity as the murine starting material.

(4) NANOBODY Methods

Antibodies against Aβ can also be produced via the Nanobody™ methods (Ablynx N.V.). Nanobodies are antibody-derived therapeutic proteins that contain the properties of naturally-occurring heavy chain antibodies. Nanobodies can function as a single, relatively small, functional antigen-binding structural unit, domain or protein. The Nanobody™ technology was originally developed following the discovery that camelidae (camels and llamas) possess fully functional antibodies that lack light chains. These heavy-chain antibodies contain a single variable domain (VHH) and two constant domains (CH2 and CH3). VHH is used to distinguish them from the heavy chain variable domains that are present in conventional 4-chain antibodies (which are referred to as “VH domains”). The cloned and isolated VHH domain is a stable polypeptide harboring the full antigen-binding capacity of the original heavy-chain antibody. VHH domains and nanobodies can also be engineered into multivalent and multispecific formats. Nanobodies with an amino acid sequence that corresponds to the amino acid sequence of a naturally occurring VHH domain can be humanized, i.e., by replacing one or more amino acid residues in the amino acid sequence of the naturally occurring VHH sequence (and in particular in the framework sequences) by one or more of the amino acid residues that occur at the corresponding position(s) in a VH domain from a conventional 4-chain antibody from a human being. For details, see e.g., US 20050130266, US 20040253638, WO/2006/040153, US 20050214857, WO/2006/079372, or WO/2006/122825, each of which is incorporated herein by reference for all purposes.

v. Selection of Constant Region

The heavy and light chain variable regions of chimeric, humanized, or human antibodies can be linked to at least a portion of a human constant region. The choice of constant region depends, in part, whether antibody-dependent complement and/or cellular mediated toxicity is desired. For example, isotopes IgG1 and IgG3 have complement activity and isotypes IgG2 and IgG4 do not. Choice of isotype can also affect passage of antibody into the brain. Human isotype IgG1 is preferred. Light chain constant regions can be lambda or kappa. Antibodies can be expressed as tetramers containing two light and two heavy chains, as separate heavy chains, light chains, as Fab, Fab′ F(ab′)₂, and Fv, or as single chain antibodies in which heavy and light chain variable domains are linked through a spacer.

vi. Expression of Recombinant Antibodies

Chimeric, humanized and human antibodies are typically produced by recombinant expression. Recombinant polynucleotide constructs typically include an expression control sequence operably linked to the coding sequences of antibody chains, including naturally associated or heterologous promoter regions. Preferably, the expression control sequences are eukaryotic promoter systems in vectors capable of transforming or transfecting eukaryotic host cells. Once the vector has been incorporated into the appropriate host, the host is maintained under conditions suitable for high-level expression of the nucleotide sequences, and the collection and purification of the crossreacting antibodies.

These expression vectors are typically replicable in the host organisms either as episomes or as an integral part of the host chromosomal DNA. Commonly, expression vectors contain selection markers, e.g., ampicillin-resistance or hygromycin-resistance, to permit detection of those cells transformed with the desired DNA sequences.

E. coli is one prokaryotic host particularly useful for cloning the DNA sequences of the present invention. Microbes, such as yeast, are also useful for expression. Saccharomyces is a preferred yeast host, with suitable vectors having expression control sequences, an origin of replication, termination sequences and the like as desired. Typical promoters include 3-phosphoglycerate kinase and other glycolytic enzymes. Inducible yeast promoters include, among others, promoters from alcohol dehydrogenase, isocytochrome C, and enzymes responsible for maltose and galactose utilization.

Mammalian cells are a preferred host for expressing nucleotide segments encoding immunoglobulins or fragments thereof. See Winnacker, From Genes to Clones, (VCH Publishers, NY, 1987). A number of suitable host cell lines capable of secreting intact heterologous proteins have been developed in the art, and include CHO cell lines, various COS cell lines, HeLa cells, L cells, human embryonic kidney cell, and myeloma cell lines. Preferably, the cells are nonhuman. Expression vectors for these cells can include expression control sequences, such as an origin of replication, a promoter, an enhancer (Queen et al., Immunol. Rev. 89:49 (1986)), and necessary processing information sites, such as ribosome binding sites, RNA splice sites, polyadenylation sites, and transcriptional terminator sequences. Preferred expression control sequences are promoters derived from endogenous genes, cytomegalovirus, SV40, adenovirus, bovine papillomavirus, and the like. See Co et al., J. Immunol. 148:1149 (1992).

Alternatively, antibody coding sequences can be incorporated in transgenes for introduction into the genome of a transgenic animal and subsequent expression in the milk of the transgenic animal (see, e.g., U.S. Pat. No. 5,741,957, U.S. Pat. No. 5,304,489, U.S. Pat. No. 5,849,992). Suitable transgenes include coding sequences for light and/or heavy chains in operable linkage with a promoter and enhancer from a mammary gland specific gene, such as casein or beta lactoglobulin.

The vectors containing the DNA segments of interest can be transferred into the host cell by well-known methods, depending on the type of cellular host. For example, calcium chloride transfection is commonly utilized for prokaryotic cells, whereas calcium phosphate treatment, electroporation, lipofection, biolistics or viral-based transfection can be used for other cellular hosts. Other methods used to transform mammalian cells include the use of polybrene, protoplast fusion, liposomes, electroporation, and microinjection (see generally, Sambrook et al., supra). For production of transgenic animals, transgenes can be microinjected into fertilized oocytes, or can be incorporated into the genome of embryonic stem cells, and the nuclei of such cells transferred into enucleated oocytes.

Once expressed, antibodies can be purified according to standard procedures of the art, including HPLC purification, column chromatography, gel electrophoresis and the like (see generally, Scopes, Protein Purification (Springer-Verlag, NY, 1982)).

3D6 or a chimeric or humanized form thereof is a preferred antibody (see U.S. Patent Publication No. 20030165496A1, U.S. Patent Publication No. 20040087777A1, International Patent Publication No. WO 02/46237A3 and International Patent Publication No. WO 04/080419A2). Description of 3D6 can also be found, for example, in International Patent Publication No. WO 02/088306A2 and International Patent Publication No. WO02/088307A2. Additional 3D6 antibodies are described in U.S. patent application Ser. No. 11/303,478 and International Application No. PCT/US05/45614. 3D6 is a monoclonal antibody (mAb) that specifically binds to an N-terminal epitope located in the human β-amyloid peptide, specifically, residues 1-5. A cell line producing the 3D6 monoclonal antibody (RB96 3D6.32.2.4) was deposited with the American Type Culture Collection (ATCC), Manassas, Va. 20108, USA on Apr. 8, 2003 under the terms of the Budapest Treaty and has deposit number PTA-5130.

Bapineuzumab means a humanized 3D6 antibody comprising a light chain having a mature variable region having the amino acid sequence designated SEQ ID NO: 1 and a heavy chain having a mature variable region having the amino acid sequence designated SEQ ID NO: 2 is shown below.

Humanized 3D6 Light Chain Variable Region

(SEQ ID NO:1)

Asp Val Val Met Thr Gln Ser Pro Leu Ser Leu Pro
Val Thr Pro Gly Glu Pro Ala Ser Ile Ser Cys Lys
Ser Ser Gln Ser Leu Leu Asp Ser Asp Gly Lys Thr
Tyr Leu Asn Trp Leu Leu Gln Lys Pro Gly Gln Ser
Pro Gln Arg Leu Ile Tyr Leu Val Ser Lys Leu Asp
Ser Gly Val Pro Asp Arg Phe Ser Gly Ser Gly Ser
Gly Thr Asp Phe Thr Leu Lys Ile Ser Arg Val Glu
Ala Glu Asp Val Gly Val Tyr Tyr Cys Trp Gln Gly
Thr His Phe Pro Arg Thr Phe Gly Gln Gly Thr Lys
Val Glu Ile Lys

Humanized 3D6 Heavy Chain Variable Region

(SEQ ID NO:2)

Glu Val Gln Leu Leu Glu Ser Gly Gly Gly Leu Val
Gln Pro Gly Gly Ser Leu Arg Leu Ser Cys Ala Ala
Ser Gly Phe Thr Phe Ser Asn Tyr Gly Met Ser Trp
Val Arg Gln Ala Pro Gly Lys Gly Leu Glu Trp Val
Ala Ser Ile Arg Ser Gly Gly Gly Arg Thr Tyr Tyr
Ser Asp Asn Val Lys Gly Arg Phe Thr Ile Ser Arg
Asp Asn Ser Lys Asn Thr Leu Tyr Leu Gln Met Asn
Ser Leu Arg Ala Glu Asp Thr Ala Val Tyr Tyr Cys
Val Arg Tyr Asp His Tyr Ser Gly Ser Ser Asp Tyr
Trp Gly Gln Gly Thr Leu Val Thr Val Ser Ser

Bapineuzumab is known as AAB-001.

A second version of humanized 3D6 antibody comprising a light chain having a mature variable region having the amino acid sequence designated SEQ ID NO: 3 and a heavy chain having a mature variable region having the amino acid sequence designated SEQ ID NO: 4 is shown below.

Humanized 3D6 Light Chain Variable Region

(SEQ ID NO:3)

Tyr Val Val Met Thr Gln Ser Pro Leu Ser Leu Pro
Val Thr Pro Gly Glu Pro Ala Ser Ile Ser Cys Lys
Ser Ser Gln Ser Leu Leu Asp Ser Asp Gly Lys Thr
Tyr Leu Asn Trp Leu Leu Gln Lys Pro Gly Gln Ser
Pro Gln Arg Leu Ile Tyr Leu Val Ser Lys Leu Asp
Ser Gly Val Pro Asp Arg Phe Ser Gly Ser Gly Ser
Gly Thr Asp Phe Thr Leu Lys Ile Ser Arg Val Glu
Ala Glu Asp Val Gly Val Tyr Tyr Cys Trp Gln Gly
Thr His Phe Pro Arg Thr Phe Gly Gln Gly Thr Lys
Val Glu Ile Lys

Humanized 3D6 Heavy Chain Variable Region

(SEQ ID NO:4)

Glu Val Gln Leu Leu Glu Ser Gly Gly Gly Leu Val
Gln Pro Gly Gly Ser Leu Arg Leu Ser Cys Ala Ala
Ser Gly Phe Thr Phe Ser Asn Tyr Gly Met Ser Trp
Val Arg Gln Ala Pro Gly Lys Gly Leu Glu Trp Val
Ala Ser Ile Arg Ser Gly Gly Gly Arg Thr Tyr Tyr
Ser Asp Asn Val Lys Gly Arg Phe Thr Ile Ser Arg
Asp Asn Ala Lys Asn Ser Leu Tyr Leu Gln Met Asn
Ser Leu Arg Ala Glu Asp Thr Ala Leu Tyr Tyr Cys
Val Arg Tyr Asp His Tyr Ser Gly Ser Ser Asp Tyr
Trp Gly Gln Gly Thr Leu Val Thr Val Ser Ser

A third version of humanized 3D6 antibody comprising a light chain having the amino acid sequence designated SEQ ID NO: 5 and a heavy chain having the amino acid sequence designated SEQ ID NO: 6 is described in US 2005/0090649 A1 published on Apr. 28, 2005, which is incorporated herein by reference for all purposes. Humanized 3D6 Light Chain

(SEQ ID NO: 5)

Asp Val Val Met Thr Gln Ser Pro Leu Ser Leu Pro
Val Thr Leu Gly Gln Pro Ala Ser Ile Ser Cys Lys
Ser Ser Gln Ser Leu Leu Asp Ser Asp Gly Lys Thr
Tyr Leu Asn Trp Leu Gln Gln Arg Pro Gly Gln Ser
Pro Arg Arg Leu Ile Tyr Leu Val Ser Lys Leu Asp
Ser Gly Val Pro Asp Arg Phe Ser Gly Ser Gly Ser
Gly Thr Asp Phe Thr Leu Lys Ile Ser Arg Val Glu
Ala Glu Asp Val Gly Val Tyr Tyr Cys Trp Gln Gly
Thr His Phe Pro Arg Thr Phe Gly Gly Gly Thr Lys
Val Glu Ile Lys Arg Thr Val Ala Ala Pro Ser Val
Phe Ile Phe Pro Pro Ser Asp Glu Gln Leu Lys Ser
Gly Thr Ala Ser Val Val Cys Leu Leu Asn Asn Phe
Tyr Pro Arg Glu Ala Lys Val Gln Trp Lys Val Asp
Asn Ala Leu Gln Ser Gly Asn Ser Gln Glu Ser Val
Thr Glu Gln Asp Ser Lys Asp Ser Thr Tyr Ser Leu
Ser Ser Thr Leu Thr Leu Ser Lys Ala Asp Tyr Glu
Lys His Lys Val Tyr Ala Cys Glu Val Thr His Gln
Gly Leu Ser Ser Pro Val Thr Lys Ser Phe Asn Arg
Gly Glu Cys

Humanized 3D6 Heavy Chain

(SEQ ID NO: 6)

Gln Val Gln Leu Val Gln Ser Gly Gly Gly Leu Val
Gln Pro Gly Gly Ser Leu Arg Leu Ser Cys Ala Gly
Ser Gly Phe Thr Phe Ser Asn Tyr Gly Met Ser Trp
Val Arg Gln Ala Pro Gly Lys Gly Leu Glu Trp Val
Ala Ser Ile Arg Ser Gly Gly Gly Arg Thr Tyr Tyr
Ser Asp Asn Val Lys Gly Arg Phe Thr Ile Ser Arg
Glu Asn Ala Lys Asn Ser Leu Tyr Leu Gln Met Asn
Ser Leu Arg Ala Glu Asp Thr Ala Val Tyr Tyr Cys
Val Arg Tyr Asp His Tyr Ser Gly Ser Ser Asp Tyr
Trp Gly Gln Gly Thr Leu Val Thr Val Ser Ser Ala
Ser Thr Lys Gly Pro Ser Val Phe Pro Leu Ala Pro
Ser Ser Lys Ser Thr Ser Gly Gly Thr Ala Ala Leu
Gly Cys Leu Val Lys Asp Tyr Phe Pro Gln Pro Val
Thr Val Ser Trp Asn Ser Gly Ala Leu Thr Ser Gly
Val His Thr Phe Pro Ala Val Leu Gln Ser Ser Gly
Leu Tyr Ser Leu Ser Ser Val Val Thr Val Pro Ser
Ser Ser Leu Gly Thr Gln Thr Tyr Ile Cys Asn Val
Asn His Lys Pro Ser Asn Thr Lys Val Asp Lys Lys
Val Glu Pro Lys Ser Cys Asp Lys Thr His Thr Cys
Pro Pro Cys Pro Ala Pro Gln Leu Leu Gly Gly Pro
Ser Val Phe Leu Phe Pro Pro Lys Pro Lys Asp Thr
Leu Met Ile Ser Arg Thr Pro Glu Val Thr Cys Val
Val Val Asp Val Ser His Glu Asp Pro Glu Val Lys
Phe Asn Trp Tyr Val Asp Gly Val Glu Val His Asn
Ala Lys Thr Lys Pro Arg Glu Glu Gln Tyr Asn Ser
Thr Tyr Arg Val Val Ser Val Leu Thr Val Leu His
Gln Asp Trp Leu Asn Gly Lys Glu Tyr Lys Cys Lys
Val Ser Asn Lys Ala Leu Pro Ala Pro Ile Glu Lys
Thr Ile Ser Lys Ala Lys Gly Gln Pro Arg Glu Pro
Gln Val Tyr Thr Leu Pro Pro Ser Arg Asp Glu Leu
Thr Lys Asn Gln Val Ser Leu Thr Cys Leu Val Lys
Gly Phe Tyr Pro Ser Asp Ile Ala Val Glu Trp Glu
Ser Asn Gly Gln Pro Glu Asn Asn Tyr Lys Thr Thr
Pro Pro Val Leu Asp Ser Asp Gly Ser Phe Phe Leu
Tyr Ser Lys Leu Thr Val Asp Lys Ser Arg Trp Gln
Gln Gly Asn Val Phe Ser Cys Ser Val Met His Glu
Ala Leu His Asn His Tyr Thr Gln Lys Ser Leu Ser
Leu Ser Pro Gly Lys

12A11 or a chimeric or humanized or nanobody form thereof is a preferred antibody. The 12A11 antibody or a variant thereof, is described in U.S. Patent Publication No. 20050118651, U.S. Patent Publication No. 20060198851, International Patent Publication No. WO 04/108895, and International Patent Publication No. WO 06/066089, all of which are incorporated by reference in their entirety herein for all purposes. 12A11 is a mAb that specifically binds to an N-terminal epitope located in the human β-amyloid peptide, specifically, residues 3-7. A cell line producing the 12A11 monoclonal antibody was deposited at the ATCC (American Type Culture Collection, 10801 University Boulevard, Manassas, Va. 20110-2209) on Dec. 12, 2005 and has the ATCC accession number PTA-7271.

A first version of the humanized 12A11 antibody comprising a light chain having the amino acid sequence designated SEQ ID NO: 7 and a heavy chain having the amino acid sequence designated SEQ ID NO: 8 (version 1) is described in US 20050118651 A1 published on Jun. 2, 2005, which is incorporated herein by reference for all purposes.

Humanized 12A11 Light Chain

(SEQ ID NO: 7)

Asp Val Val Met Thr Gln Ser Pro Leu Ser Leu Pro
Val Thr Pro Gly Glu Pro Ala Ser Ile Ser Cys Arg
Ser Ser Gln Ser Ile Val His Ser Asn Gly Asn Thr
Tyr Leu Glu Trp Tyr Leu Gln Lys Pro Gly Gln Ser
Pro Gln Leu Leu Ile Tyr Lys Val Ser Asn Arg Phe
Ser Gly Val Pro Asp Arg Phe Ser Gly Ser Gly Ser
Gly Thr Asp Phe Thr Leu Lys Ile Ser Arg Val Glu
Ala Glu Asp Val Gly Val Tyr Tyr Cys Phe Gln Ser
Ser His Val Pro Leu Thr Phe Gly Gln Gly Thr Lys
Leu Glu Ile Lys

Humanized 12A11 Heavy Chain (version 1)

(SEQ ID NO: 8)

Gln Val Gln Leu Val Glu Ser Gly Gly Gly Val Val
Gln Pro Gly Arg Ser Leu Arg Leu Ser Cys Ala Phe
Ser Gly Phe Ser Leu Ser Thr Ser Gly Met Ser Val
Gly Trp Ile Arg Gln Ala Pro Gly Lys Gly Leu Glu
Trp Leu Ala His Ile Trp Trp Asp Asp Asp Lys Tyr
Tyr Asn Pro Ser Leu Lys Ser Arg Leu Thr Ile Ser
Lys Asp Thr Ser Lys Asn Thr Val Tyr Leu Gln Met
Asn Ser Leu Arg Ala Glu Asp Thr Ala Val Tyr Tyr
Cys Ala Arg Arg Thr Thr Thr Ala Asp Tyr Phe Ala
Tyr Trp Gly Gln Gly Thr Thr Val Thr Val Ser Ser

A second version of the humanized 12A11 antibody comprising a light chain having the amino acid sequence designated SEQ ID NO: 7 and a heavy chain having the amino acid sequence designated SEQ ID NO: 9 (version 2) is described in US 20050118651 A1 published on Jun. 2, 2005, which is incorporated herein by reference for all purposes. Humanized 12A 11 Light Chain

(SEQ ID NO. 7)

Asp Val Val Met Thr Gln Ser Pro Leu Ser Leu Pro
Val Thr Pro Gly Glu Pro Ala Ser Ile Ser Cys Arg
Ser Ser Gln Ser Ile Val His Ser Asn Gly Asn Thr
Tyr Leu Glu Trp Tyr Leu Gln Lys Pro Gly Gln Ser
Pro Gln Leu Leu Ile Tyr Lys Val Ser Asn Arg Phe
Ser Gly Val Pro Asp Arg Phe Ser Gly Ser Gly Ser
Gly Thr Asp Phe Thr Leu Lys Ile Ser Arg Val Glu
Ala Glu Asp Val Gly Val Tyr Tyr Cys Phe Gln Ser
Ser His Val Pro Leu Thr Phe Gly Gln Gly Thr Lys
Leu Glu Ile Lys

Humanized 12A11 Heavy Chain (version 2)

(SEQ ID No: 9)

Gln Val Gln Leu Val Glu Ser Gly Gly Gly Val Val
Gln Pro Gly Arg Ser Leu Arg Leu Ser Cys Ala Phe
Ser Gly Phe Thr Leu Ser Thr Ser Gly Met Ser Val
Gly Trp Ile Arg Gln Ala Pro Gly Lys Gly Leu Glu
Trp Val Ala His Ile Trp Trp Asp Asp Asp Lys Tyr
Tyr Asn Pro Ser Leu Lys Ser Arg Phe Thr Ile Ser
Lys Asp Thr Ser Lys Asn Thr Leu Tyr Leu Gln Met
Asn Ser Leu Arg Ala Glu Asp Thr Ala Val Tyr Tyr
Cys Ala Arg Arg Thr Thr Thr Ala Asp Tyr Phe Ala
Tyr Trp Gly Gln Gly Thr Thr Val Thr Val Ser Ser

A third version of the humanized 12A11 antibody comprising a light chain having the amino acid sequence designated SEQ ID NO: 7 and a heavy chain having the amino acid sequence designated SEQ ID NO: 10 (version 2.1) is described in US 20050118651 A1 published on Jun. 2, 2005, which is incorporated herein by reference for all purposes.

Humanized 12A11 Light Chain

(SEQ ID NO: 7)

Asp Val Val Met Thr Gln Ser Pro Leu Ser Leu Pro
Val Thr Pro Gly Glu Pro Ala Ser Ile Ser Cys Arg
Ser Ser Gln Ser Ile Val His Ser Asn Gly Asn Thr
Tyr Leu Glu Trp Tyr Leu Gln Lys Pro Gly Gln Ser
Pro Gln Leu Leu Ile Tyr Lys Val Ser Asn Arg Phe
Ser Gly Val Pro Asp Arg Phe Ser Gly Ser Gly Ser
Gly Thr Asp Phe Thr Leu Lys Ile Ser Arg Val Glu
Ala Glu Asp Val Gly Val Tyr Tyr Cys Phe Gln Ser
Ser His Val Pro Leu Thr Phe Gly Gln Gly Thr Lys
Leu Glu Ile Lys

Humanized 12A11 Heavy Chain (version 2.1)

(SEQ ID NO: 10)

Gln Val Gln Leu Val Glu Ser Gly Gly Gly Val Val
Gln Pro Gly Arg Ser Leu Arg Leu Ser Cys Ala Phe
Ser Gly Phe Thr Leu Ser Thr Ser Gly Met Ser Val
Gly Trp Ile Arg Gln Ala Pro Gly Lys Gly Leu Glu
Trp Val Ala His Ile Trp Trp Asp Asp Asp Lys Tyr
Tyr Asn Pro Ser Leu Lys Ser Arg Phe Thr Ile Ser
Lys Asp Asn Ser Lys Asn Thr Leu Tyr Leu Gln Met
Asn Ser Leu Arg Ala Glu Asp Thr Ala Val Tyr Tyr
Cys Ala Arg Arg Thr Thr Thr Ala Asp Tyr Phe Ala
Tyr Trp Gly Gln Gly Thr Thr Val Thr Val Ser Ser

A fourth version of the humanized 12A11 antibody comprising a light chain having the amino acid sequence designated SEQ ID NO: 7 and a heavy chain having the amino acid sequence designated SEQ ID NO: 11 (version 3) is described in US 20050118651 A1 published on Jun. 2, 2005, which is incorporated herein by reference for all purposes.

Humanized 12A11 Light Chain

(SEQ ID NO: 7)

Asp Val Val Met Thr Gln Ser Pro Leu Ser Leu Pro
Val Thr Pro Gly Glu Pro Ala Ser Ile Ser Cys Arg
Ser Ser Gln Ser Ile Val His Ser Asn Gly Asn Thr
Tyr Leu Glu Trp Tyr Leu Gln Lys Pro Gly Gln Ser
Pro Gln Leu Leu Ile Tyr Lys Val Ser Asn Arg Phe
Ser Gly Val Pro Asp Arg Phe Ser Gly Ser Gly Ser
Gly Thr Asp Phe Thr Leu Lys Ile Ser Arg Val Glu
Ala Glu Asp Val Gly Val Tyr Tyr Cys Phe Gln Ser
Ser His Val Pro Leu Thr Phe Gly Gln Gly Thr Lys
Leu Glu Ile Lys

Humanized 12A11 Heavy Chain (version 3)

(SEQ ID NO: 11)

Gln Val Gln Leu Val Glu Ser Gly Gly Gly Val Val
Gln Pro Gly Arg Ser Leu Arg Leu Ser Cys Ala Phe
Ser Gly Phe Thr Leu Ser Thr Ser Gly Met Ser Val
Gly Trp Ile Arg Gln Ala Pro Gly Lys Gly Leu Glu
Trp Val Ala His Ile Trp Trp Asp Asp Asp Lys Tyr
Tyr Asn Pro Ser Leu Lys Ser Arg Phe Thr Ile Ser
Arg Asp Asn Ser Lys Asn Thr Leu Tyr Leu Gln Met
Asn Ser Leu Arg Ala Glu Asp Thr Ala Val Tyr Tyr
Cys Ala Arg Arg Thr Thr Thr Ala Asp Tyr Phe Ala
Tyr Trp Gly Gln Gly Thr Thr Val Thr Val Ser Ser

A fifth version of the humanized 12A11 antibody comprising a light chain having the amino acid sequence designated SEQ ID NO: 7 and a heavy chain having the amino acid sequence designated SEQ ID NO: 12 (version 4.1) is described in U.S. Pat. No. 20050118651 A1 published on Jun. 2, 2005, which is incorporated herein by reference for all purposes.

Humanized 12A11 Light Chain

(SEQ ID NO. 7)

Asp Val Val Met Thr Gln Ser Pro Leu Ser Leu Pro
Val Thr Pro Gly Glu Pro Ala Ser Ile Ser Cys Arg
Ser Ser Gln Ser Ile Val His Ser Asn Gly Asn Thr
Tyr Leu Glu Trp Tyr Leu Gln Lys Pro Gly Gln Ser
Pro Gln Leu Leu Ile Tyr Lys Val Ser Asn Arg Phe
Ser Gly Val Pro Asp Arg Phe Ser Gly Ser Gly Ser
Gly Thr Asp Phe Thr Leu Lys Ile Ser Arg Val Glu
Ala Glu Asp Val Gly Val Tyr Tyr Cys Phe Gln Ser
Ser His Val Pro Leu Thr Phe Gly Gln Gly Thr Lys
Leu Glu Ile Lys

Humanized 12A11 Heavy Chain (version 4.1)

(SEQ ID NO: 12)

Gln Val Gln Leu Val Glu Ser Gly Gly Gly Val Val
Gln Pro Gly Arg Ser Leu Arg Leu Ser Cys Ala Phe
Ser Gly Phe Thr Leu Ser Thr Ser Gly Met Ser Val
Gly Trp Ile Arg Gln Ala Pro Gly Lys Gly Leu Glu
Trp Leu Ala His Ile Trp Trp Asp Asp Asp Lys Tyr
Tyr Asn Pro Ser Leu Lys Ser Arg Leu Thr Ile Ser
Lys Asp Thr Ser Lys Asn Thr Val Tyr Leu Gln Met
Asn Ser Leu Arg Ala Glu Asp Thr Ala Val Tyr Tyr
Cys Ala Arg Arg Thr Thr Thr Ala Asp Tyr Phe Ala
Tyr Trp Gly Gln Gly Thr Thr Val Thr Val Ser Ser

A sixth version of the humanized 12A11 antibody comprising a light chain having the amino acid sequence designated SEQ ID NO: 7 and a heavy chain having the amino acid sequence designated SEQ ID NO: 13 (version 4.2) is described in US 20050118651 A1 published on Jun. 2, 2005, which is incorporated herein by reference for all purposes.

Humanized 12A11 Light Chain

(SEQ ID NO: 7)

Asp Val Val Met Thr Gln Ser Pro Leu Ser Leu Pro
Val Thr Pro Gly Glu Pro Ala Ser Ile Ser Cys Arg
Ser Ser Gln Ser Ile Val His Ser Asn Gly Asn Thr
Tyr Leu Glu Trp Tyr Leu Gln Lys Pro Gly Gln Ser
Pro Gln Leu Leu Ile Tyr Lys Val Ser Asn Arg Phe
Ser Gly Val Pro Asp Arg Phe Ser Gly Ser Gly Ser
Gly Thr Asp Phe Thr Leu Lys Ile Ser Arg Val Glu
Ala Glu Asp Val Gly Val Tyr Tyr Cys Phe Gln Ser
Ser His Val Pro Leu Thr Phe Gly Gln Gly Thr Lys
Leu Glu Ile Lys

Humanized 12A11 Heavy Chain (version 4.2)

(SEQ ID NO: 13)

Gln Val Gln Leu Val Glu Ser Gly Gly Gly Val Val
Gln Pro Gly Arg Ser Leu Arg Leu Ser Cys Ala Phe
Ser Gly Phe Ser Leu Ser Thr Ser Gly Met Ser Val
Gly Trp Ile Arg Gln Ala Pro Gly Lys Gly Leu Glu
Trp Val Ala His Ile Trp Trp Asp Asp Asp Lys Tyr
Tyr Asn Pro Ser Leu Lys Ser Arg Leu Thr Ile Ser
Lys Asp Thr Ser Lys Asn Thr Val Tyr Leu Gln Met
Asn Ser Leu Arg Ala Glu Asp Thr Ala Val Tyr Tyr
Cys Ala Arg Arg Thr Thr Thr Ala Asp Tyr Phe Ala
Tyr Trp Gly Gln Gly Thr Thr Val Thr Val Ser Ser

An seventh version of the humanized 12A11 antibody comprising a light chain having the amino acid sequence designated SEQ ID NO: 7 and a heavy chain having the amino acid sequence designated SEQ ID NO: 14 (version 4.3) is described in US 20050118651 A1 published on Jun. 2, 2005, which is incorporated herein by reference for all purposes.

Humanized 12A11 Light Chain

(SEQ ID NO: 7)

Asp Val Val Met Thr Gln Ser Pro Leu Ser Leu Pro
Val Thr Pro Gly Glu Pro Ala Ser Ile Ser Cys Arg
Ser Ser Gln Ser Ile Val His Ser Asn Gly Asn Thr
Tyr Leu Glu Trp Tyr Leu Gln Lys Pro Gly Gln Ser
Pro Gln Leu Leu Ile Tyr Lys Val Ser Asn Arg Phe
Ser Gly Val Pro Asp Arg Phe Ser Gly Ser Gly Ser
Gly Thr Asp Phe Thr Leu Lys Ile Ser Arg Val Glu
Ala Glu Asp Val Gly Val Tyr Tyr Cys Phe Gln Ser
Ser His Val Pro Leu Thr Phe Gly Gln Gly Thr Lys
Leu Glu Ile Lys

Humanized 12A11 Heavy Chain (version 4.3)

(SEQ ID NO: 14)

Gln Val Gln Leu Val Glu Ser Gly Gly Gly Val Val
Gln Pro Gly Arg Ser Leu Arg Leu Ser Cys Ala Phe
Ser Gly Phe Ser Leu Ser Thr Ser Gly Met Ser Val
Gly Trp Ile Arg Gln Ala Pro Gly Lys Gly Leu Glu
Trp Leu Ala His Ile Trp Trp Asp Asp Asp Lys Tyr
Tyr Asn Pro Ser Leu Lys Ser Arg Phe Thr Ile Ser
Lys Asp Thr Ser Lys Asn Thr Val Tyr Leu Gln Met
Asn Ser Leu Arg Ala Glu Asp Thr Ala Val Tyr Tyr
Cys Ala Arg Arg Thr Thr Thr Ala Asp Tyr Phe Ala
Tyr Trp Gly Gln Gly Thr Thr Val Thr Val Ser Ser

A eighth version of the humanized 12A11 antibody comprising a light chain having the amino acid sequence designated SEQ ID NO: 7 and a heavy chain having the amino acid sequence designated SEQ ID NO: 15 (version 4.4) is described in US 20050118651 A1 published on Jun. 2, 2005, which is incorporated herein by reference for all purposes.

Humanized 12A 11 Light Chain

(SEQ ID NO: 7)

Asp Val Val Met Thr Gln Ser Pro Leu Ser Leu Pro
Val Thr Pro Gly Glu Pro Ala Ser Ile Ser Cys Arg
Ser Ser Gln Ser Ile Val His Ser Asn Gly Asn Thr
Tyr Leu Glu Trp Tyr Leu Gln Lys Pro Gly Gln Ser
Pro Gln Leu Leu Ile Tyr Lys Val Ser Asn Arg Phe
Ser Gly Val Pro Asp Arg Phe Ser Gly Ser Gly Ser
Gly Thr Asp Phe Thr Leu Lys Ile Ser Arg Val Glu
Ala Glu Asp Val Gly Val Tyr Tyr Cys Phe Gln Ser
Ser His Val Pro Leu Thr Phe Gly Gln Gly Thr Lys
Leu Glu Ile Lys

Humanized 12A11 Heavy Chain (version 4.4)

(SEQ ID NO: 15)

Gln Val Gln Leu Val Glu Ser Gly Gly Gly Val Val
Gln Pro Gly Arg Ser Leu Arg Leu Ser Cys Ala Phe
Ser Gly Phe Ser Leu Ser Thr Ser Gly Met Ser Val
Gly Trp Ile Arg Gln Ala Pro Gly Lys Gly Leu Glu
Trp Leu Ala His Ile Trp Trp Asp Asp Asp Lys Tyr
Tyr Asn Pro Ser Leu Lys Ser Arg Leu Thr Ile Ser
Lys Asp Thr Ser Lys Asn Thr Leu Tyr Leu Gln Met
Asn Ser Leu Arg Ala Glu Asp Thr Ala Val Tyr Tyr
Cys Ala Arg Arg Thr Thr Thr Ala Asp Tyr Phe Ala
Tyr Trp Gly Gln Gly Thr Thr Val Thr Val Ser Ser

A ninth version of the humanized 12A11 antibody comprising a light chain having the amino acid sequence designated SEQ ID NO: 7 and a heavy chain having the amino acid sequence designated SEQ ID NO: 16 (version 5.1) is described in US 20050118651 A1 published on Jun. 2, 2005, which is incorporated herein by reference for all purposes.

Humanized 12A 11 Light Chain

(SEQ ID. NO: 7)

Asp Val Val Met Thr Gln Ser Pro Leu Ser Leu Pro
Val Thr Pro Gly Glu Pro Ala Ser Ile Ser Cys Arg
Ser Ser Gln Ser Ile Val His Ser Asn Gly Asn Thr
Tyr Leu Glu Trp Tyr Leu Gln Lys Pro Gly Gln Ser
Pro Gln Leu Leu Ile Tyr Lys Val Ser Asn Arg Phe
Ser Gly Val Pro Asp Arg Phe Ser Gly Ser Gly Ser
Gly Thr Asp Phe Thr Leu Lys Ile Ser Arg Val Glu
Ala Glu Asp Val Gly Val Tyr Tyr Cys Phe Gln Ser
Ser His Val Pro Leu Thr Phe Gly Gln Gly Thr Lys
Leu Glu Ile Lys

Humanized 12A11 Heavy Chain (version 5.1)

(SEQ ID NO: 16)

Gln Val Gln Leu Val Glu Ser Gly Gly Gly Val Val
Gln Pro Gly Arg Ser Leu Arg Leu Ser Cys Ala Phe
Ser Gly Phe Thr Leu Ser Thr Ser Gly Met Ser Val
Gly Trp Ile Arg Gln Ala Pro Gly Lys Gly Leu Glu
Trp Val Ala His Ile Trp Trp Asp Asp Asp Lys Tyr
Tyr Asn Pro Ser Leu Lys Ser Arg Leu Thr Ile Ser
Lys Asp Thr Ser Lys Asn Thr Val Tyr Leu Gln Met
Asn Ser Leu Arg Ala Glu Asp Thr Ala Val Tyr Tyr
Cys Ala Arg Arg Thr Thr Thr Ala Asp Tyr Phe Ala
Tyr Trp Gly Gln Gly Thr Thr Val Thr Val Ser Ser

A tenth version of the humanized 12A11 antibody comprising a light chain having the amino acid sequence designated SEQ ID NO: 7 and a heavy chain having the amino acid sequence designated SEQ ID NO: 17 (version 5.2) is described in US 20050118651 A1 published on Jun. 2, 2005, which is incorporated herein by reference for all purposes.

Humanized 12A11 Light Chain

(SEQ ID NO: 7)

Asp Val Val Met Thr Gln Ser Pro Leu Ser Leu Pro
Val Thr Pro Gly Glu Pro Ala Ser Ile Ser Cys Arg
Ser Ser Gln Ser Ile Val His Ser Asn Gly Asn Thr
Tyr Leu Glu Trp Tyr Leu Gln Lys Pro Gly Gln Ser
Pro Gln Leu Leu Ile Tyr Lys Val Ser Asn Arg Phe
Ser Gly Val Pro Asp Arg Phe Ser Gly Ser Gly Ser
Gly Thr Asp Phe Thr Leu Lys Ile Ser Arg Val Glu
Ala Glu Asp Val Gly Val Tyr Tyr Cys Phe Gln Ser
Ser His Val Pro Leu Thr Phe Gly Gln Gly Thr Lys
Leu Glu Ile Lys

Humanized 12A11 Heavy Chain (version 5.2)

(SEQ ID NO: 17)

Gln Val Gln Leu Val Glu Ser Gly Gly Gly Val Val
Gln Pro Gly Arg Ser Leu Arg Leu Ser Cys Ala Phe
Ser Gly Phe Thr Leu Ser Thr Ser Gly Met Ser Val
Gly Trp Ile Arg Gln Ala Pro Gly Lys Gly Leu Glu
Trp Leu Ala His Ile Trp Trp Asp Asp Asp Lys Tyr
Tyr Asn Pro Ser Leu Lys Ser Arg Phe Thr Ile Ser
Lys Asp Thr Ser Lys Asn Thr Val Tyr Leu Gln Met
Asn Ser Leu Arg Ala Glu Asp Thr Ala Val Tyr Tyr
Cys Ala Arg Arg Thr Thr Thr Ala Asp Tyr Phe Ala
Tyr Trp Gly Gln Gly Thr Thr Val Thr Val Ser Ser

An eleventh version of the humanized 12A11 antibody comprising a light chain having the amino acid sequence designated SEQ ID NO: 7 and a heavy chain having the amino acid sequence designated SEQ ID NO: 18 (version 5.3) is described in US 20050118651 A1 published on Jun. 2, 2005, which is incorporated herein by reference for all purposes.

Humanized 12A11 Light Chain

(SEQ ID NO: 7)

Asp Val Val Met Thr Gln Ser Pro Leu Ser Leu Pro
Val Thr Pro Gly Glu Pro Ala Ser Ile Ser Cys Arg
Ser Ser Gln Ser Ile Val His Ser Asn Gly Asn Thr
Tyr Leu Glu Trp Tyr Leu Gln Lys Pro Gly Gln Ser
Pro Gln Leu Leu Ile Tyr Lys Val Ser Asn Arg Phe
Ser Gly Val Pro Asp Arg Phe Ser Gly Ser Gly Ser
Gly Thr Asp Phe Thr Leu Lys Ile Ser Arg Val Glu
Ala Glu Asp Val Gly Val Tyr Tyr Cys Phe Gln Ser
Ser His Val Pro Leu Thr Phe Gly Gln Gly Thr Lys
Leu Glu Ile Lys

Humanized 12A11 Heavy Chain (Version 5.3)

(SEQ ID NO: 18)

Gln Val Gln Leu Val Glu Ser Gly Gly Gly Val Val
Gln Pro Gly Arg Ser Leu Arg Leu Ser Cys Ala Phe
Ser Gly Phe Thr Leu Ser Thr Ser Gly Met Ser Val
Gly Trp Ile Arg Gln Ala Pro Gly Lys Gly Leu Glu
Trp Leu Ala His Ile Trp Trp Asp Asp Asp Lys Tyr
Tyr Asn Pro Ser Leu Lys Ser Arg Leu Thr Ile Ser
Lys Asp Thr Ser Lys Asn Thr Leu Tyr Leu Gln Met
Asn Ser Leu Arg Ala Glu Asp Thr Ala Val Tyr Tyr
Cys Ala Arg Arg Thr Thr Thr Ala Asp Tyr Phe Ala
Tyr Trp Gly Gln Gly Thr Thr Val Thr Val Ser Ser
Val

A twelfth version of the humanized 12A11 antibody comprising a light chain having the amino acid sequence designated SEQ ID NO: 7 and a heavy chain having the amino acid sequence designated SEQ ID NO: 19 (version 5.4) is described in US 20050118651 A1 published on Jun. 2, 2005, which is incorporated herein by reference for all purposes.

Humanized 12A11 Light Chain

(SEQ ID NO: 7)

Asp Val Val Met Thr Gln Ser Pro Leu Ser Leu Pro
Val Thr Pro Gly Glu Pro Ala Ser Ile Ser Cys Arg
Ser Ser Gln Ser Ile Val His Ser Asn Gly Asn Thr
Tyr Leu Glu Trp Tyr Leu Gln Lys Pro Gly Gln Ser
Pro Gln Leu Leu Ile Tyr Lys Val Ser Asn Arg Phe
Ser Gly Val Pro Asp Arg Phe Ser Gly Ser Gly Ser
Gly Thr Asp Phe Thr Leu Lys Ile Ser Arg Val Glu
Ala Glu Asp Val Gly Val Tyr Tyr Cys Phe Gln Ser
Ser His Val Pro Leu Thr Phe Gly Gln Gly Thr Lys
Leu Glu Ile Lys

Humanized 12A11 Heavy Chain (Version 5.4)

(SEQ ID NO: 19)

Gln Val Gln Leu Val Glu Ser Gly Gly Gly Val Val
Gln Pro Gly Arg Ser Leu Arg Leu Ser Cys Ala Phe
Ser Gly Phe Ser Leu Ser Thr Ser Gly Met Ser Val
Gly Trp Ile Arg Gln Ala Pro Gly Lys Gly Leu Glu
Trp Val Ala His Ile Trp Trp Asp Asp Asp Lys Tyr
Tyr Asn Pro Ser Leu Lys Ser Arg Phe Thr Ile Ser
Lys Asp Thr Ser Lys Asn Thr Val Tyr Leu Gln Met
Asn Ser Leu Arg Ala Glu Asp Thr Ala Val Tyr Tyr
Cys Ala Arg Arg Thr Thr Thr Ala Asp Tyr Phe Ala
Tyr Trp Gly Gln Gly Thr Thr Val Thr Val Ser Ser
Val

A thirteenth version of the humanized 12A11 antibody comprising a light chain having the amino acid sequence designated SEQ ID NO: 7 and a heavy chain having the amino acid sequence designated SEQ ID NO: 20 (version 5.5) is described in US 20050118651 A1 published on Jun. 2, 2005, which is incorporated herein by reference for all purposes.

Humanized 12A11 Light Chain

(SEQ ID NO: 7)

Asp Val Val Met Thr Gln Ser Pro Leu Ser Leu Pro
Val Thr Pro Gly Glu Pro Ala Ser Ile Ser Cys Arg
Ser Ser Gln Ser Ile Val His Ser Asn Gly Asn Thr
Tyr Leu Glu Trp Tyr Leu Gln Lys Pro Gly Gln Ser
Pro Gln Leu Leu Ile Tyr Lys Val Ser Asn Arg Phe
Ser Gly Val Pro Asp Arg Phe Ser Gly Ser Gly Ser
Gly Thr Asp Phe Thr Leu Lys Ile Ser Arg Val Glu
Ala Glu Asp Val Gly Val Tyr Tyr Cys Phe Gln Ser
Ser His Val Pro Leu Thr Phe Gly Gln Gly Thr Lys
Leu Glu Ile Lys

Humanized 12A11 Heavy Chain (Version 5.5)

(SEQ ID NO: 20)

Gln Val Gln Leu Val Glu Ser Gly Gly Gly Val Val
Gln Pro Gly Arg Ser Leu Arg Leu Ser Cys Ala Phe
Ser Gly Phe Ser Leu Ser Thr Ser Gly Met Ser Val
Gly Trp Ile Arg Gln Ala Pro Gly Lys Gly Leu Glu
Trp Val Ala His Ile Trp Trp Asp Asp Asp Lys Tyr
Tyr Asn Pro Ser Leu Lys Ser Arg Leu Thr Ile Ser
Lys Asp Thr Ser Lys Asn Thr Leu Tyr Leu Gln Met
Asn Ser Leu Arg Ala Glu Asp Thr Ala Val Tyr Tyr
Cys Ala Arg Arg Thr Thr Thr Ala Asp Tyr Phe Ala
Tyr Trp Gly Gln Gly Thr Thr Val Thr Val Ser Ser

A fourteenth version of the humanized 12A11 antibody comprising a light chain having the amino acid sequence designated SEQ ID NO: 7 and a heavy chain having the amino acid sequence designated SEQ ID NO: 21 (version 5.6) is described in US 20050118651 A1 published on Jun. 2, 2005, which is incorporated herein by reference for all purposes.

Humanized 12A11 Light Chain

(SEQ ID NO: 7)

Asp Val Val Met Thr Gln Ser Pro Leu Ser Leu Pro
Val Thr Pro Gly Glu Pro Ala Ser Ile Ser Cys Arg
Ser Ser Gln Ser Ile Val His Ser Asn Gly Asn Thr
Tyr Leu Glu Trp Tyr Leu Gln Lys Pro Gly Gln Ser
Pro Gln Leu Leu Ile Tyr Lys Val Ser Asn Arg Phe
Ser Gly Val Pro Asp Arg Phe Ser Gly Ser Gly Ser
Gly Thr Asp Phe Thr Leu Lys Ile Ser Arg Val Glu
Ala Glu Asp Val Gly Val Tyr Tyr Cys Phe Gln Ser
Ser His Val Pro Leu Thr Phe Gly Gln Gly Thr Lys
Leu Glu Ile Lys

Humanized 12A11 Heavy Chain (Version 5.6)

(SEQ ID NO: 21)

Gln Val Gln Leu Val Glu Ser Gly Gly Gly Val Val
Gln Pro Gly Arg Ser Leu Arg Leu Ser Cys Ala Phe
Ser Gly Phe Ser Leu Ser Thr Ser Gly Met Ser Val
Gly Trp Ile Arg Gln Ala Pro Gly Lys Gly Leu Glu
Trp Leu Ala His Ile Trp Trp Asp Asp Asp Lys Tyr
Tyr Asn Pro Ser Leu Lys Ser Arg Phe Thr Ile Ser
Lys Asp Thr Ser Lys Asn Thr Leu Tyr Leu Gln Met
Asn Ser Leu Arg Ala Glu Asp Thr Ala Val Tyr Tyr
Cys Ala Arg Arg Thr Thr Thr Ala Asp Tyr Phe Ala
Tyr Trp Gly Gln Gly Thr Thr Val Thr Val Ser Ser

A fifteenth version of the humanized 12A11 antibody comprising a light chain having the amino acid sequence designated SEQ ID NO: 7 and a heavy chain having the amino acid sequence designated SEQ ID NO: 22 (version 6.1) is described in US 20050118651 A1 published on Jun. 2, 2005, which is incorporated herein by reference for all purposes.

Humanized 12A11 Light Chain

(SEQ ID NO: 7)

Asp Val Val Met Thr Gln Ser Pro Leu Ser Leu Pro
Val Thr Pro Gly Glu Pro Ala Ser Ile Ser Cys Arg
Ser Ser Gln Ser Ile Val His Ser Asn Gly Asn Thr
Tyr Leu Glu Trp Tyr Leu Gln Lys Pro Gly Gln Ser
Pro Gln Leu Leu Ile Tyr Lys Val Ser Asn Arg Phe
Ser Gly Val Pro Asp Arg Phe Ser Gly Ser Gly Ser
Gly Thr Asp Phe Thr Leu Lys Ile Ser Arg Val Glu
Ala Glu Asp Val Gly Val Tyr Tyr Cys Phe Gln Ser
Ser His Val Pro Leu Thr Phe Gly Gln Gly Thr Lys
Leu Glu Ile Lys

Humanized 12A11 Heavy Chain (Version 6.1)

(SEQ ID NO: 22)

Gln Val Gln Leu Val Glu Ser Gly Gly Gly Val Val
Gln Pro Gly Arg Ser Leu Arg Leu Ser Cys Ala Phe
Ser Gly Phe Thr Leu Ser Thr Ser Gly Met Ser Val
Gly Trp Ile Arg Gln Ala Pro Gly Lys Gly Leu Glu
Trp Val Ala His Ile Trp Trp Asp Asp Asp Lys Tyr
Tyr Asn Pro Ser Leu Lys Ser Arg Phe Thr Ile Ser
Lys Asp Thr Ser Lys Asn Thr Val Tyr Leu Gln Met
Asn Ser Leu Arg Ala Glu Asp Thr Ala Val Tyr Tyr
Cys Ala Arg Arg Thr Thr Thr Ala Asp Tyr Phe Ala
Tyr Trp Gly Gln Gly Thr Thr Val Thr Val Ser Ser

A sixteenth version of the humanized 12A11 antibody comprising a light chain having the amino acid sequence designated SEQ ID NO: 7 and a heavy chain having the amino acid sequence designated SEQ ID NO: 23 (version 6.2) is described in US 20050118651 A1 published on Jun. 2, 2005, which is incorporated herein by reference for all purposes.

Humanized 12A11 Light Chain

(SEQ ID NO: 7)

Asp Val Val Met Thr Gln Ser Pro Leu Ser Leu Pro
Val Thr Pro Gly Glu Pro Ala Ser Ile Ser Cys Arg
Ser Ser Gln Ser Ile Val His Ser Asn Gly Asn Thr
Tyr Leu Glu Trp Tyr Leu Gln Lys Pro Gly Gln Ser
Pro Gln Leu Leu Ile Tyr Lys Val Ser Asn Arg Phe
Ser Gly Val Pro Asp Arg Phe Ser Gly Ser Gly Ser
Gly Thr Asp Phe Thr Leu Lys Ile Ser Arg Val Glu
Ala Glu Asp Val Gly Val Tyr Tyr Cys Phe Gln Ser
Ser His Val Pro Leu Thr Phe Gly Gln Gly Thr Lys
Leu Glu Ile Lys

Humanized 12A11 Heavy Chain (Version 6.2)

(SEQ ID NO: 23)

Gln Val Gln Leu Val Glu Ser Gly Gly Gly Val Val
Gln Pro Gly Arg Ser Leu Arg Leu Ser Cys Ala Phe
Ser Gly Phe Thr Leu Ser Thr Ser Gly Met Ser Val
Gly Trp Ile Arg Gln Ala Pro Gly Lys Gly Leu Glu
Trp Val Ala His Ile Trp Trp Asp Asp Asp Lys Tyr
Tyr Asn Pro Ser Leu Lys Ser Arg Leu Thr Ile Ser
Lys Asp Thr Ser Lys Asn Thr Leu Tyr Leu Gln Met
Asn Ser Leu Arg Ala Glu Asp Thr Ala Val Tyr Tyr
Cys Ala Arg Arg Thr Thr Thr Ala Asp Tyr Phe Ala
Tyr Trp Gly Gln Gly Thr Thr Val Thr Val Ser Ser

A seventeenth version of the humanized 12A11 antibody comprising a light chain having the amino acid sequence designated SEQ ID NO: 7 and a heavy chain having the amino acid sequence designated SEQ ID NO: 24 (version 6.3) is described in US 20050118651 A1 published on Jun. 2, 2005, which is incorporated herein by reference for all purposes.

Humanized 12A11 Light Chain

(SEQ ID NO: 7)

Asp Val Val Met Thr Gln Ser Pro Leu Ser Leu Pro
Val Thr Pro Gly Glu Pro Ala Ser Ile Ser Cys Arg
Ser Ser Gln Ser Ile Val His Ser Asn Gly Asn Thr
Tyr Leu Glu Trp Tyr Leu Gln Lys Pro Gly Gln Ser
Pro Gln Leu Leu Ile Tyr Lys Val Ser Asn Arg Phe
Ser Gly Val Pro Asp Arg Phe Ser Gly Ser Gly Ser
Gly Thr Asp Phe Thr Leu Lys Ile Ser Arg Val Glu
Ala Glu Asp Val Gly Val Tyr Tyr Cys Phe Gln Ser
Ser His Val Pro Leu Thr Phe Gly Gln Gly Thr Lys
Leu Glu Ile Lys

Humanized 12A11 Heavy Chain (Version 6.3)

(SEQ ID NO: 24)

Gln Val Gln Leu Val Glu Ser Gly Gly Gly Val Val
Gln Pro Gly Arg Ser Leu Arg Leu Ser Cys Ala Phe
Ser Gly Phe Thr Leu Ser Thr Ser Gly Met Ser Val
Gly Trp Ile Arg Gln Ala Pro Gly Lys Gly Leu Glu
Trp Leu Ala His Ile Trp Trp Asp Asp Asp Lys Tyr
Tyr Asn Pro Ser Leu Lys Ser Arg Phe Thr Ile Ser
Lys Asp Thr Ser Lys Asn Thr Leu Tyr Leu Gln Met
Asn Ser Leu Arg Ala Glu Asp Thr Ala Val Tyr Tyr
Cys Ala Arg Arg Thr Thr Thr Ala Asp Tyr Phe Ala
Tyr Trp Gly Gln Gly Thr Thr Val Thr Val Ser Ser

A eighteenth version of the humanized 12A11 antibody comprising a light chain having the amino acid sequence designated SEQ ID NO: 7 and a heavy chain having the amino acid sequence designated SEQ ID NO: 25 (version 6.4) is described in U.S. Pat. No. 20050118651 A1 published on Jun. 2, 2005, which is incorporated herein by reference for all purposes.

Humanized 12A11 Light Chain

(SEQ ID NO: 7)

Asp Val Val Met Thr Gln Ser Pro Leu Ser Leu Pro
Val Thr Pro Gly Glu Pro Ala Ser Ile Ser Cys Arg
Ser Ser Gln Ser Ile Val His Ser Asn Gly Asn Thr
Tyr Leu Glu Trp Tyr Leu Gln Lys Pro Gly Gln Ser
Pro Gln Leu Leu Ile Tyr Lys Val Ser Asn Arg Phe
Ser Gly Val Pro Asp Arg Phe Ser Gly Ser Gly Ser
Gly Thr Asp Phe Thr Leu Lys Ile Ser Arg Val Glu
Ala Glu Asp Val Gly Val Tyr Tyr Cys Phe Gln Ser
Ser His Val Pro Leu Thr Phe Gly Gln Gly Thr Lys
Leu Glu Ile Lys

Humanized 12A11 Heavy Chain (Version 6.4)

(SEQ ID NO: 25)

Gln Val Gln Leu Val Glu Ser Gly Gly Gly Val Val
Gln Pro Gly Arg Ser Leu Arg Leu Ser Cys Ala Phe
Ser Gly Phe Ser Leu Ser Thr Ser Gly Met Ser Val
Gly Trp Ile Arg Gln Ala Pro Gly Lys Gly Leu Glu
Trp Val Ala His Ile Trp Trp Asp Asp Asp Lys Tyr
Tyr Asn Pro Ser Leu Lys Ser Arg Phe Thr Ile Ser
Lys Asp Thr Ser Lys Asn Thr Leu Tyr Leu Gln Met
Asn Ser Leu Arg Ala Glu Asp Thr Ala Val Tyr Tyr
Cys Ala Arg Arg Thr Thr Thr Ala Asp Tyr Phe Ala
Tyr Trp Gly Gln Gly Thr Thr Val Thr Val Ser Ser

A nineteenth version of the humanized 12A11 antibody comprising a light chain having the amino acid sequence designated SEQ ID NO: 7 and a heavy chain having the amino acid sequence designated SEQ ID NO: 26 (version 7) is described in US 20050118651 A1 published on Jun. 2, 2005, which is incorporated herein by reference for all purposes.

Humanized 12A11 Light Chain

(SEQ ID NO: 7)

Asp Val Val Met Thr Gln Ser Pro Leu Ser Leu Pro
Val Thr Pro Gly Glu Pro Ala Ser Ile Ser Cys Arg
Ser Ser Gln Ser Ile Val His Ser Asn Gly Asn Thr
Tyr Leu Glu Trp Tyr Leu Gln Lys Pro Gly Gln Ser
Pro Gln Leu Leu Ile Tyr Lys Val Ser Asn Arg Phe
Ser Gly Val Pro Asp Arg Phe Ser Gly Ser Gly Ser
Gly Thr Asp Phe Thr Leu Lys Ile Ser Arg Val Glu
Ala Glu Asp Val Gly Val Tyr Tyr Cys Phe Gln Ser
Ser His Val Pro Leu Thr Phe Gly Gln Gly Thr Lys
Leu Glu Ile Lys

Humanized 12A11 Heavy Chain (Version 7)

(SEQ ID NO: 26)

Gln Val Gln Leu Val Glu Ser Gly Gly Gly Val Val
Gln Pro Gly Arg Ser Leu Arg Leu Ser Cys Ala Phe
Ser Gly Phe Thr Leu Ser Thr Ser Gly Met Ser Val
Gly Trp Val Arg Gln Ala Pro Gly Lys Gly Leu Glu
Trp Leu Ala His Ile Trp Trp Asp Asp Asp Lys Tyr
Tyr Asn Pro Ser Leu Lys Ser Arg Leu Thr Ile Ser
Lys Asp Thr Ser Lys Asn Thr Val Tyr Leu Gln Met
Asn Ser Leu Arg Ala Glu Asp Thr Ala Val Tyr Tyr
Cys Ala Arg Arg Thr Thr Thr Ala Asp Tyr Phe Ala
Tyr Trp Gly Gln Gly Thr Thr Val Thr Val Ser Ser

A twentieth version of the humanized 12A11 antibody comprising a light chain having the amino acid sequence designated SEQ ID NO: 7 and a heavy chain having the amino acid sequence designated SEQ ID NO: 27 (version 8) is described in US 20050118651 A1 published on Jun. 2, 2005, which is incorporated herein by reference for all purposes.

Humanized 12A11 Light Chain

(SEQ ID NO: 7)

Asp Val Val Met Thr Gln Ser Pro Leu Ser Leu Pro
Val Thr Pro Gly Glu Pro Ala Ser Ile Ser Cys Arg
Ser Ser Gln Ser Ile Val His Ser Asn Gly Asn Thr
Tyr Leu Glu Trp Tyr Leu Gln Lys Pro Gly Gln Ser
Pro Gln Leu Leu Ile Tyr Lys Val Ser Asn Arg Phe
Ser Gly Val Pro Asp Arg Phe Ser Gly Ser Gly Ser
Gly Thr Asp Phe Thr Leu Lys Ile Ser Arg Val Glu
Ala Glu Asp Val Gly Val Tyr Tyr Cys Phe Gln Ser
Ser His Val Pro Leu Thr Phe Gly Gln Gly Thr Lys
Leu Glu Ile Lys

Humanized 12A11 Heavy Chain (Version 8)

(SEQ ID NO: 8)

Gln Val Gln Leu Val Glu Ser Gly Gly Gly Val Val
Gln Pro Gly Arg Ser Leu Arg Leu Ser Cys Ala Phe
Ser Gly Phe Ser Leu Ser Thr Ser Gly Met Ser Val
Gly Trp Ile Arg Gln Ala Pro Gly Lys Gly Leu Glu
Trp Leu Ala His Ile Trp Trp Asp Asp Asp Lys Tyr
Tyr Asn Pro Ser Leu Lys Ser Arg Leu Thr Ile Ser
Lys Asp Asn Ser Lys Asn Thr Val Tyr Leu Gln Met
Asn Ser Leu Arg Ala Glu Asp Thr Ala Val Tyr Tyr
Cys Ala Arg Arg Thr Thr Thr Ala Asp Tyr Phe Ala
Tyr Trp Gly Gln Gly Thr Thr Val Thr Val Ser Ser

Any of the antibodies described above can be produced with different isotypes or mutant isotypes to control the extent of binding to different Fc receptors. Antibodies lacking an Fc region (e.g., Fab fragments) lack binding to Fc receptors. Selection of isotype also affects binding to Fc receptors. The respective affinities of various human IgG isotypes for the three Fcγ receptors, FcRI, FcRII, and FcRIII, have been determined (see Ravetch & Kinet, Annu. Rev. Immunol. 9, 457 (1991)). FcRI is a high affinity receptor that binds to IgGs in monomeric form, and the latter two are low affinity receptors that bind IgGs only in multimeric form. In general, both IgG1 and IgG3 have significant binding activity to all three receptors, IgG4 to FcRI, and IgG2 to only one type of FcRII called IIa_{LR} (see Parren et al., J. Immunol. 148, 695 (1992). Therefore, human isotype IgG1 is usually selected for stronger binding to Fcγ receptors is desired, and IgG2 is usually selected for weaker binding.

Mutations on adjacent or close sites in the hinge link region (e.g., replacing residues 234, 235, 236 and/or 237 with another residue) in all of the isotypes reduce affinity for Fcγ receptors, particularly FcγRI receptor. Optionally, positions 234, 236 and/or 237 are substituted with alanine and position 235 with glutamine. (See, e.g., U.S. Pat. No. 5,624,821)Position 236 is missing in the human IgG2 isotype. Exemplary segments of amino acids for positions 234, 235 and 237 for human IgG2 are ala ala gly, val ala ala, ala ala ala, val glu ala, and ala glu ala. A preferred combination of mutants is L234A, L235A, and G237A for human isotype IgG1. A particular preferred antibody is bapineuzumab having human isotype IgG and these three mutations of the Fc region. Other substitutions that decrease finding to Fc gamma receptors are an E233P mutation (particularly in mouse IgG1) and D265A.

Amino acids in the constant region are numbered by alignment with the human antibody EU (see Cunningham et al., J. Biol. Chem., 9, 3161 (1970)). That is, the heavy and light chains of an antibody are aligned with the heavy and light chains of EU to maximize amino acid sequence identity and each amino acid in the antibody is assigned the same number as the corresponding amino acid in EU. The EU numbering system is conventional (see generally, Kabat et al, Sequences of Protein of Immunological Interest, NIH Publication No. 91-3242, US Department of Health and Human Services (1991)).

The affinity of an antibody for complement component Clq can be altered by mutating at least one of the amino acid residues 318, 320, and 322 of the heavy chain has been changed to a residue having a different side chain. Other suitable alterations for altering, e.g., reducing or abolishing specific Clq-binding to an antibody include changing any one of residues 318 (Glu), 320 (Lys) and 322 (Lys), to Ala. Clq binding activity can be abolished by replacing any one of the three specified residues with a residue having an inappropriate functionality on its side chain. It is not necessary to replace the ionic residues only with Ala to abolish Clq binding. It is also possible to use other alkyl-substituted non-ionic residues, such as Gly, Ile, Leu, or Val, or such aromatic non-polar residues as Phe, Tyr, Trp and Pro in place of any one of the three residues in order to abolish Clq binding. In addition, it is also be possible to use such polar non-ionic residues as Ser, Thr, Cys, and Met in place of residues 320 and 322, but not 318, to abolish Clq binding activity. Replacement of the 318 (Glu) residue by a polar residue may modify but not abolish Clq binding activity. Replacing residue 297 (Asn) with Ala results in removal of lytic activity while only slightly reducing (about three fold weaker) affinity for Clq. This alteration destroys the glycosylation site and the presence of carbohydrate that is required for complement activation. Any other substitution at this site also destroy the glycosylation site.

III. Patients Amenable to (CAA) Treatment Regimes

Cerebral amyloid angiopathy is also known as congophilic angiopathy or cerebrovascular amyloidosis. It is a disease of small blood vessels in the brain in which deposits of amyloid protein in the vessel walls may lead to stroke, brain hemorrhage, white matter ischemia or dementia. Amyloid protein resembles a starch and is deposited in tissues during the course of certain chronic diseases.

CAA may affect patients over age 45, but is most common in patients over age 65, and becomes more common with increasing age. Men and women are equally affected. In some cases, CAA is sporadic but it may also be inherited as an autosomal dominant condition (a form of inheritance in which only one copy of a gene coding for a disease need be present for that disease to be expressed; if either parent has the disease, a child has a 50% chance of inheriting the disease). CAA is responsible for 5-20% of brain hemorrhage, and up to 30% of lobar hemorrhages localized to one lobe of the brain. CAA may be found during an autopsy in over one-third of persons over age 60, even though they may not have had brain hemorrhage, stroke, or other manifestations of the disease during life. In Alzheimer's disease, CAA is more common than in the general population, and may occur in more than 80% of Alzheimer's patients over age 60.

The cause of amyloid deposits in blood vessels in the brain in sporadic CAA is not known. In hereditary CAA, genetic defects, typically on chromosome 21, allow accumulation of amyloid, a protein made up of units called beta-pleated sheet fibrils. The fibrils tend to clump together, so that the amyloid cannot be dissolved and builds up in the brain blood vessel walls. One form of amyloid fibril subunit proteins is the amyloid beta protein.

Amyloid deposits may destroy endothelial or smooth muscle cells, or both endothelial or smooth muscle cells, or cause inflammation in the blood vessel wall and may also cause the blood vessel to break more easily. Bleeding into the brain may also occur as tiny blood vessels carrying amyloid deposits become heavier and more brittle, and are therefore more likely to burst with minor trauma or with fluctuating blood pressure. Aneurysms, or ballooning of the blood vessel wall, may develop, and may also rupture as the stretched wall becomes thinner and is under more pressure.

The most common form of CAA is the sporadic form associated with aging. This type of CAA usually causes lobar hemorrhage, which may recur in different lobes of the brain. The frontal lobe (behind the forehead) and parietal lobe (behind the frontal lobe) are most often affected; the temporal lobe (near the temple) and occipital lobe (at the back of the brain) are affected less often; and the cerebellum (under the occipital lobe) is rarely affected. Approximately 10-50% of hemorrhages in sporadic CAA involve more than one lobe.

Symptoms of lobar hemorrhage in CAA include sudden onset of headache, neurologic symptoms such as weakness, sensory loss, visual changes, or speech problems, depending on which lobe is involved; and decreased level of consciousness (a patient who is difficult to arouse), nausea, and vomiting. Sporadic CAA may be associated with symptoms unrelated to lobar hemorrhage. Petechial hemorrhages (tiny hemorrhages involving many small vessels) may produce recurrent, brief neurologic symptoms secondary to seizures or decreased blood flow, or may produce rapidly progressive dementia (loss of memory and other brain functions) that worsens in distinct steps rather than gradually. Over 40% of patients with hemorrhage secondary to CAA also have dementia.

Genetic factors play a role in certain types of CAA and in diseases associated with CAA:
Dutch type of hereditary cerebral hemorrhage with amyloidosis (build up of amyloid protein in blood vessels): autosomal dominant, with a genetic mutation involving the amyloid precursor protein. Onset is at age 40-60 with headaches, brain hemorrhage often in the parietal lobe, strokes, and dementia. More than half of patients die from their first hemorrhage. Patients with the Dutch type of CAA may produce an abnormal anticoagulant, or blood thinner, which makes hemorrhage more likely.
Flemish type of hereditary cerebral hemorrhage with amyloidosis: autosomal dominant, with a mutation involving the amyloid precursor protein. Symptoms include brain hemorrhage or dementia.
Familial Alzheimer's disease: autosomal dominant, comprising 5-10% of all Alzheimer's disease cases (a brain disease in which death of nerve cells leads to progressive dementia).
Down Syndrome: caused by trisomy 21 (three rather than two copies of chromosome 21), causing excess amyloid precursor protein gene. Children with Down syndrome are mentally handicapped and may have heart problems.
Icelandic type of hereditary cerebral hemorrhage with amyloidosis: autosomal dominant, with mutation in the gene coding for cystatin C. Symptoms often begin at age 30-40 with multiple brain hemorrhages, dementia, paralysis (weakness), and death in 10-20 years. Headache occurs in more than half of patients, and seizures occur in one-quarter. Unlike most other forms of CAA, most hemorrhages involve the basal ganglia deep within the brain. (Basal ganglia are islands of tissues in the cerebellum part of the brain.)

Familial oculo-leptomeningeal amyloidosis: autosomal dominant with unknown gene defect(s), described in Japanese, Italian, and North American families. Symptoms can include dementia, ataxia (problems with coordination), spasticity (limb stiffness), strokes, seizures, peripheral neuropathy (disease affecting the nerves supplying the limbs), migraine, spinal cord problems, blindness, and deafness. Brain hemorrhage is rare as the amyloid protein is deposited in blood vessels in the eye and meninges (brain coverings), but not in the brain itself. In Italian families with the disease, patients may be affected as early as 20-30 years of age.

British type of familial amyloidosis: autosomal dominant with unknown gene defect(s), associated with progressive dementia, spasticity, and ataxia. Brain stem, spinal cord, and cerebellum all exhibit amyloid deposits, but hemorrhage typically does not occur.

In some methods, a patient has CAA and is free of symptoms of Alzheimer's or other disease amenable to treatment by antibodies to Aβ or agents capable of inducing the same. In other methods, the patient has concurrent CAA and Alzheimer's or other disease amenable to treatment by antibodies to Aβ or agents capable of inducing the same. In other methods, a patient is free of CAA and Alzheimer's disease and any other disease amenable to treatment by antibodies to Aβ or agents capable of inducing the same.

In asymptomatic patients, treatment can begin at any age (e.g., 10, 20, or 30). Usually, however, it is not necessary to begin treatment until a patient reaches 40, 50, 60, or 70. Treatment typically entails multiple dosages over a period of time. Treatment can be monitored by assaying antibody, or activated T-cell or B-cell responses to the therapeutic agent (antibody to Aβ, or a fragment thereof) over time. If the response falls, a booster dosage is indicated.

Optionally, presence of absence of symptoms, signs or risk factors of a disease is determined before beginning treatment.

### IV. Diagnosis and Monitoring of CAA Patients

As in most neurologic diseases, diagnosis is made most often from the patient's history, with careful inquiry into family history and the patient's onset and pattern of symptoms, as well as neurologic examination. Brain computed tomography scan (CT) or magnetic resonance imaging (MRI) may identify lobar hemorrhage, stroke, or petechial hemorrhages, and are important in excluding arteriovenous malformation, brain tumor, or other causes of hemorrhage. Angiography (x-ray study of the interior of blood vessels and the heart) is not helpful in diagnosis of CAA, but may be needed to exclude aneurysm. Brain biopsy (surgical removal of a small piece of brain tissue) may show characteristic amyloid deposits. If diagnosis is uncertain, biopsy may be needed to rule out conditions which are potentially treatable. Lumbar puncture to examine cerebrospinal fluid proteins may show characteristic abnormalities.

CAA with hemorrhage must be distinguished from other types of brain hemorrhage. In CAA, hemorrhage typically occurs in the lobar region, often ruptures into the subarachnoid space between the brain and its coverings, and occurs at night. In hemorrhage related to high blood pressure, hemorrhage is usually deeper within the brain, ruptures into the ventricles or cavities deep inside the brain, and occurs during daytime activities. Other causes of brain hemorrhage are arteriovenous malformations, trauma, aneurysms, bleeding into a brain tumor, vasculitis (inflammation of blood vessels), or bleeding disorders. Patients can be monitored for cerebral microhemorrhage by MRI and/or for vascular amyloid removal by positron emission tomography (PET) scan. Patients amenable to treatment include individuals at risk of a CAA but not showing symptoms, as well as patients presently showing symptoms.

### V. Treatment Regimes

In prophylactic applications, pharmaceutical compositions or medicaments are administered to a patient susceptible to, or otherwise at risk of a CAA in regime comprising an amount and frequency of administration of the composition or medicament sufficient to eliminate or reduce the risk, lessen the severity, or delay the outset of the disease, including physiological, biochemical, histologic and/or behavioral symptoms of the disease, its complications and intermediate pathological phenotypes presenting during development of the disease. In therapeutic applications, compositions or medicates are administered to a patient suspected of, or already suffering from such a disease in a regime comprising an amount and frequency of administration of the composition sufficient to cure, or at least partially arrest, the symptoms of the disease (physiological, biochemical, histologic and/or behavioral), including its complications and intermediate pathological phenotypes in development of the disease. An amount adequate to accomplish therapeutic or prophylactic treatment is defined as a therapeutically- or prophylactically-effective dose. A combination of amount and dosage frequency adequate to accomplish therapeutic or prophylactic treatment is defined as a therapeutically or prophylatically-effective regime. In both prophylactic and therapeutic regimes, agents are usually administered in several dosages until a sufficient immune response has been achieved. Typically, the immune response is monitored and repeated dosages are given if the immune response starts to wane.

Effective doses of the compositions of the present invention, for the treatment of the above described conditions vary depending upon many different factors, including means of administration, target site, physiological state of the patient, whether the patient is human or an animal, other medications administered, and whether treatment is prophylactic or therapeutic. Usually, the patient is a human but nonhuman mammals including transgenic mammals can also be treated. Treatment dosages need to be titrated to optimize safety and efficacy. The amount of immunogen depends on whether adjuvant is also administered, with higher dosages being required in the absence of adjuvant. The amount of an immunogen for administration sometimes varies from 1-500 μg per patient and more usually from 5-500 μg per injection for human administration. Typically about 10, 20, 50 or 100 μg is used for each human injection. The mass of immunogen also depends on the mass ratio of immunogenic epitope within the immunogen to the mass of immunogen as a whole. Typically, 10⁻³ to 10⁻⁵ micromoles of immunogenic epitope are used for microgram of immunogen. The timing of injections can vary significantly from once a day, to once a year, to once a decade. On any given day that a dosage of immunogen is given, the dosage is greater than 1 μg/patient and usually greater than 10 μg/patient if adjuvant is also administered, and greater than 10 μg/patient and usually greater than 100 μg/patient in the absence of adjuvant. A typical regimen consists of an immunization followed by booster injections at time intervals, such as 6 week intervals. Another regimen consists of an immunization followed by booster injections 1, 2 and 12 months later. Another regimen entails an injection every two months for life. Alternatively, booster injections can be on an irregular basis as indicated by monitoring of immune response.

For passive immunization with an antibody, the dosage regime is usually 0.01 to 5 mg/kg, of the host body weight. In particular, the dosage ranges from about 0.5 to less than 5 mg/kg, and more usually 0.5 to 3 mg/kg, of the host body weight. For example dosages can be less than 5 mg/kg body weight or 1.5 mg/kg body weight or within the range of 0.5 to 1.5 mg/kg, preferably at least 1.5 mg/kg. Subjects can be administered such doses daily, on alternative days, weekly or according to any other schedule determined by empirical analysis. An exemplary treatment entails administration in multiple dosages over a prolonged period, for example, of at least six months. Additional exemplary treatment regimes entail administration once per every two weeks or once a month or once every 3 to 6 months.

Exemplary passive dosage schedules include 1.5-3 mg/kg or 1.5 mg/kg every thirteen weeks. Agents of the invention are usually administered on multiple occasions. Intervals between single dosages can be weekly, monthly, every thirteen weeks, or yearly. Intervals can also be irregular as indicated by measuring blood levels of antibody to Aβ in the patient.

In some methods, dosage is adjusted to achieve a plasma antibody concentration of 1-1000 μg/ml and in some methods 25-300 μg/ml. Alternatively, antibody can be administered as a sustained release formulation, in which case less frequent administration is required. Dosage and frequency vary depending on the half-life of the antibody in the patient. In general, human antibodies show the longest half-life, followed by humanized antibodies, chimeric antibodies, and nonhuman antibodies.

Preferred regimes for administering antibodies specific for the N-terminus of Aβ achieve an average serum concentration of administered antibody of 1-15 μg/ml in a patient. The serum concentration can be determined by actual measurement or predicted from standard pharmacokinetics (e.g., WinNonline Version 4.0.1 (Pharsight Corporation, Cary, USA)) based on the amount of antibody administered, frequency of administration, route of administration and antibody half-life. The average antibody concentration in the serum is preferably within a range of 1-10, 1-5 or 2-4 μg/ml.

For intravenous administration, doses of 0.1 to 5 mg/kg of antibody administered between monthly and quarterly (every 13 weeks are preferred). For quarterly administration, the dose is preferably in a range of 0.5-3, 0.5-2 or 0.5-1.5 mg/kg. Preferred doses of antibody for monthly intravenous administration occur in the range of 0.1-1.0 mg/kg antibody or preferably 0.5-1.0 mg/kg antibody.

For more frequent dosing, e.g., from weekly to monthly dosing, subcutaneous administration is preferred. The doses used for subcutaneous dosing are usually in the range of 0.1 to 0.6 mg/kg or 0.01-0.35 mg/kg, preferably, 0.05-0.25 mg/kg. For weekly or biweekly dosing, the dose is preferably in the range of 0.015-0.2 mg/kg, or 0.05-0.15 mg/kg. For weekly dosing, the dose is preferably 0.05 to 0.07 mg/kg, e.g., 0.06 mg/kg. For biweekly dosing, the dose is preferably 0.1 to 0.15 mg/kg. For monthly dosing, the dose is preferably 0.1 to 0.3 mg/kg or 2 mg/kg. Monthly dosing includes dosing by the calendar month or lunar month (i.e., every four weeks).

The treatment regime is usually continued so that the average serum concentrations of antibody described above are maintained for at least six months or a year, and sometimes for life. The serum concentration can be measured at any time during treatment and the dose and/or frequency of administration increased if the average concentration falls beneath a target range or the dose and/or frequency decreased if the average concentration falls above a target range.

Although determining optimal plasma concentrations of antibody is useful in determining a dosage regime or optimizing dosage in an individual patient, in practice once an effective dosage regime in terms of mg/kg or mg and frequency of administration has been determined, the same dosage regime can be used on many other patients without the need for detailed calculation or measurement of patient titers. Thus, any of the above mentioned dosages and treatment regimes can be used irrespective whether a titer is measured or predicted in a particular patient. For example, one suitable regime is intravenous administration at monthly intervals with a dose in range of 0.1-1.0 mg/kg antibody or preferably 0.5-1.0 mg/kg antibody. For subcutaneous dosing the dose used is usually in the range of 0.01-0.6 mg/kg or 0.01-0.35 mg/kg, preferably, 0.05-0.25 mg/kg. For weekly or biweekly dosing, the dose is preferably in the range of 0.015-0.2 mg/kg, or 0.05-0.15 mg/kg. For weekly dosing, the dose is preferably 0.05 to 0.07 mg/kg, e.g., 0.06 mg/kg. For biweekly dosing, the dose is preferably 0.1 to 0.15 mg/kg. For monthly dosing, the dose is preferably 0.1 to 0.3 mg/kg or 2 mg/kg.

Here as elsewhere in the application, dosages expressed in mg/kg can be converted to absolute mass dosages by multiplying by the mass of a typical patient (e.g., 70 or 75 kg) typically rounding to a whole number. Expressed in terms of absolute mass, antibodies are usually administered at a dose of 1-40 mg at a frequency of between weekly and monthly. Preferred ranges are 5-25 mg or 2.5-15 mg at a frequency of weekly to monthly. For weekly to biweekly administration, the dose is often 1-12 mg or 2.5 to 10 mg. For weekly administration, the dose is often 2.5 to 5 mg or 4-5 mg. For biweekly administration, the dose can be 7-10 mg. The mass of antibody packaged for administration in unit doses is usually round to whole number, such as 1, 5, 10, 20, 30, 40, 50, 75 or 100 mg.

The dosage and frequency of administration can vary depending on whether the treatment is prophylactic or therapeutic. In prophylactic applications, compositions containing the present antibodies or a cocktail thereof are administered to a patient not already in the disease state to enhance the patient's resistance. Such an amount is defined to be a “prophylactic effective dose.” In this use, the precise amounts again depend upon the patient's state of health and general immunity, but generally range from 0.1 to 25 mg per dose, especially 0.5 to 2.5 mg per dose. A relatively low dosage is administered at relatively infrequent intervals over a long period of time. Some patients continue to receive treatment for the rest of their lives.

In therapeutic applications, a relatively high dosage (e.g., from about 10 to 250 mg of antibody per dose, with dosages of from 5 to 25 mg being more commonly used) at relatively short intervals is sometimes required until progression of the disease is reduced or terminated, and preferably until the patient shows partial or complete amelioration of symptoms of disease. Thereafter, the patent can be administered a prophylactic regime.

Agents of the invention can optionally be administered in combination with other agents that are at least partly effective in treatment of amyloidogenic disease. In the case of CAA, in which amyloid deposits occur in the brain vasculature, agents of the invention can also be administered in conjunction with other agents that increase passage of the agents of the invention across the blood-brain barrier.

Doses for nucleic acids encoding immunogens range from about 10 ng to 1 g, 100 ng to 100 mg, 1 μg to 10 mg, or 30-300 μg DNA per patient. Doses for infectious viral vectors vary from 10-100, or more, virions per dose.

Agents for inducing an immune response can be administered by parenteral, topical, intravenous, oral, subcutaneous, intraarterial, intracranial, intrathecal, intraperitoneal, intranasal or intramuscular means for prophylactic and/or therapeutic treatment. The most typical route of administration of an immunogenic agent is subcutaneous although other routes can be equally effective. The next most common route is intramuscular injection. This type of injection is most typically performed in the arm or leg muscles. In some methods, agents are injected directly into a particular tissue where deposits have accumulated, for example intracranial injection. Intramuscular injection or intravenous infusion are preferred for administration of antibody. In some methods, particular therapeutic antibodies are injected directly into the cranium. In some methods, antibodies are administered as a sustained release composition or device, such as a Medipad™ device.

As noted above, agents inducing an immunogenic response against Aβ respectively can be administered in combination. The agents can be combined in a single preparation or kit for simultaneous, sequential or separate use. The agents can occupy separate vials in the preparation or kit or can be combined in a single vial. These agents of the invention can optionally be administered in combination with other agents that are at least partly effective in treatment of CAA. The glycosaminoglycan-mimetic CEREBRILL (Neurochem) is currently in clinical trials for treatment of CAA. Most patients with CAA should be counseled to avoid agents that “thin the blood” or interfere with blood clotting. The medicine with the strongest effect on blood clotting (and thus the riskiest for CAA patients) is warfarin (also known by its trade name “Coumadin”). Other medicines that have weaker effects on the blood are aspirin, ticlopidine (“Ticlid”), clopidogrel (“Plavix”), and most of the anti-inflammatory medications such as ibuprofen. Also it is usually prudent to monitor the blood pressure after a patient recovers from a bleeding stroke and maintain it in the normal range. Seizures, or recurrent neurologic symptoms thought to be seizures, should be treated with anti-epileptic drugs, although Depakote (sodium valproate) should be avoided because of its antiplatelet effect. Anti-epileptic drugs are sometimes given to patients with large lobar hemorrhage in an attempt to prevent seizures, although the benefit of this is unclear. Surgery may be needed to remove brain hemorrhage. CAA may be rarely associated with cerebral vasculitis, or inflammation of the blood vessel walls. In these cases treatment with steroids or immune system suppressants may be helpful.

Immunogenic agents of the invention, such as peptides, are sometimes administered in combination with an adjuvant. A variety of adjuvants can be used in combination with a peptide to elicit an immune response. Preferred adjuvants augment the intrinsic response to an immunogen without causing conformational changes in the immunogen that affect the qualitative form of the response. Preferred adjuvants include aluminum hydroxide and aluminum phosphate, 3 De-O-acylated monophosphoryl lipid A (MPL™) (see GB 2220211 (RIBI ImmunoChem Research Inc., Hamilton, Mont., now part of Corixa). Stimulon™ QS-21 is a triterpene glycoside or saponin isolated from the bark of the Quillaja Saponaria Molina tree found in South America (see Kensil et al., Vaccine Design: The Subunit and Adjuvant Approach (eds. Powell & Newman, Plenum Press, NY, 1995); U.S. Pat. No. 5,057,540, Aquila BioPharmaceuticals, Framingham, Mass. Other adjuvants are oil in water emulsions (such as squalene or peanut oil), optionally in combination with immune stimulants, such as monophosphoryl lipid A (see Stoute et al., N. Engl. J. Med. 336, 86-91 (1997)), pluronic polymers, and killed mycobacteria. Another adjuvant is CpG (WO 98/40100). Alternatively, Aβ can be coupled to an adjuvant. However, such coupling should not substantially change the conformation of the immunogenalphaso as to affect the nature of the immune response thereto. Adjuvants can be administered as a component of a therapeutic composition with an active agent or can be administered separately, before, concurrently with, or after administration of the therapeutic agent.

A preferred class of adjuvants is aluminum salts (alum), such as alum hydroxide, alum phosphate, alum sulfate. Such adjuvants can be used with or without other specific immunostimulating agents such as MPL or 3-DMP, QS-21, polymeric or monomeric amino acids such as polyglutamic acid or polylysine. Another class of adjuvants is oil-in-water emulsion formulations. Such adjuvants can be used with or without other specific immunostimulating agents such as muramyl peptides (e.g., N-acetylmuramyl-L-threonyl-D-isoglutamine (thr-MDP), N-acetyl-normuramyl-L-alanyl-D-isoglutamine (nor-MDP), N-acetylmuramyl-L-alanyl-D-isoglutaminyl-L-alanine-2-(1′-2′dipalmitoyl-sn-glycero-3-hydroxyphosphoryloxy)-ethylamine (MTP-PE), N-acetylglucsaminyl-N-acetylmuramyl-N-D-isoglu-L-Ala-dipalmitoxy propylamide (DTP-DPP) Theramide™), or other bacterial cell wall components. Oil-in-water emulsions include (a) MF59 (WO 90/14837), containing 5% Squalene, 0.5% Tween 80, and 0.5% Span 85 (optionally containing various amounts of MTP-PE) formulated into submicron particles using a microfluidizer such as Model 110Y microfluidizer (Microfluidics, Newton Mass.), (b) SAF, containing 10% Squalene, 0.4% Tween 80, 5% pluronic-blocked polymer L1 21, and thr-MDP, either microfluidized into a submicron emulsion or vortexed to generate a larger particle size emulsion, and (c) Ribi™ adjuvant system (RAS), (Ribi ImmunoChem, Hamilton, Mont.) containing 2% squalene, 0.2% Tween 80, and one or more bacterial cell wall components from the group consisting of monophosphoryllipid A (MPL), trehalose dimycolate (TDM), and cell wall skeleton (CWS), preferably MPL+CWS (Detox™).

Another class of preferred adjuvants is saponin adjuvants, such as Stimulon™ (QS-21, Aquila, Framingham, Mass.) or particles generated therefrom such as ISCOMs (immunostimulating complexes) and ISCOMATRIX. Other adjuvants include RC-529, GM-CSF and pharmaceutically acceptable grades of Incomplete Freund's Adjuvant (IFA) (sold under the trade name of Montanide). Other adjuvants include cytokines, such as interleukins (e.g., IL-1, IL-2, IL-4, IL-6, IL-1 2, IL-1 3, and IL-1 5), macrophage colony stimulating factor (M-CSF), granulocyte-macrophage colony stimulating factor (GM-CSF), and tumor necrosis factor (TNF). Another class of adjuvants is glycolipid analogues including N-glycosylamides, N-glycosylureas and N-glycosylcarbamates, each of which is substituted in the sugar residue by an amino acid, as immuno-modulators or adjuvants (see U.S. Pat. No. 4,855,283). Heat shock proteins, e.g., HSP70 and HSP90, may also be used as adjuvants.

An adjuvant can be administered with an immunogen as a single composition, or can be administered before, concurrent with or after administration of the immunogen. Immunogen and adjuvant can be packaged and supplied in the same vial or can be packaged in separate vials and mixed before use. Immunogen and adjuvant are typically packaged with a label indicating the intended therapeutic application. If immunogen and adjuvant are packaged separately, the packaging typically includes instructions for mixing before use. The choice of an adjuvant and/or carrier depends on the stability of the immunogenic formulation containing the adjuvant, the route of administration, the dosing schedule, the efficacy of the adjuvant for the species being vaccinated, and, in humans, a pharmaceutically acceptable adjuvant is one that has been approved or is approvable for human administration by pertinent regulatory bodies. For example, Complete Freund's adjuvant is not suitable for human administration. Alum, MPL and QS-21 are preferred. Optionally, two or more different adjuvants can be used simultaneously. Preferred combinations include alum with MPL, alum with QS-21, MPL with QS-21, MPL or RC-529 with GM-CSF, and alum, QS-21 and MPL together. Also, Incomplete Freund's adjuvant can be used (Chang et al., Advanced Drug Delivery Reviews 32, 173-186 (1998)), optionally in combination with any of alum, QS-21, and MPL and all combinations thereof.

Agents of the invention are often administered as pharmaceutical compositions comprising an active therapeutic agent, i.e., and a variety of other pharmaceutically acceptable components. See Remington's Pharmaceutical Science (15th ed., Mack Publishing Company, Easton, Pa., 1980). The preferred form depends on the intended mode of administration and therapeutic application. The compositions can also include, depending on the formulation desired, pharmaceutically-acceptable, non-toxic carriers or diluents, which are defined as vehicles commonly used to formulate pharmaceutical compositions for animal or human administration. The diluent is selected so as not to affect the biological activity of the combination. Examples of such diluents are distilled water, physiological phosphate-buffered saline, Ringer's solutions, dextrose solution, and Hank's solution. In addition, the pharmaceutical composition or formulation may also include other carriers, adjuvants, or nontoxic, nontherapeutic, nonimmunogenic stabilizers and the like.

Pharmaceutical compositions can also include large, slowly metabolized macromolecules such as proteins, polysaccharides such as chitosan, polylactic acids, polyglycolic acids and copolymers (such as latex functionalized Sepharose™, agarose, cellulose, and the like), polymeric amino acids, amino acid copolymers, and lipid aggregates (such as oil droplets or liposomes). Additionally, these carriers can function as immunostimulating agents (i.e., adjuvants).

For parenteral administration, agents of the invention can be administered as injectable dosages of a solution or suspension of the substance in a physiologically acceptable diluent with a pharmaceutical carrier that can be a sterile liquid such as water oils, saline, glycerol, or ethanol. Additionally, auxiliary substances, such as wetting or emulsifying agents, surfactants, pH buffering substances and the like can be present in compositions. Other components of pharmaceutical compositions are those of petroleum, animal, vegetable, or synthetic origin, for example, peanut oil, soybean oil, and mineral oil. In general, glycols such as propylene glycol or polyethylene glycol are preferred liquid carriers, particularly for injectable solutions. Antibodies can be administered in the form of a depot injection or implant preparation which can be formulated in such a manner as to permit a sustained release of the active ingredient. An exemplary composition comprises monoclonal antibody at 5 mg/mL, formulated in aqueous buffer consisting of 50 mM L-histidine, 150 mM NaCl, adjusted to pH 6.0 with HCl. Compositions for parenteral administration are typically substantially sterile, substantially isotonic and manufactured under GMP conditions of the FDA or similar body. For example, compositions containing biologics are typically sterilized by filter sterilization. Compositions can be formulated for single dose administration.

Typically, compositions are prepared as injectables, either as liquid solutions or suspensions; solid forms suitable for solution in, or suspension in, liquid vehicles prior to injection can also be prepared. The preparation also can be emulsified or encapsulated in liposomes or micro particles such as polylactide, polyglycolide, or copolymer for enhanced adjuvant effect, as discussed above (see Langer, Science 249, 1527 (1990) and Hanes, Advanced Drug Delivery Reviews 28, 97-119 (1997). The agents of this invention can be administered in the form of a depot injection or implant preparation, which can be formulated in such a manner as to permit a sustained or pulsatile release of the active ingredient.

Additional formulations suitable for other modes of administration include oral, intranasal, and pulmonary formulations, suppositories, and transdermal applications.

For suppositories, binders and carriers include, for example, polyalkylene glycols or triglycerides; such suppositories can be formed from mixtures containing the active ingredient in the range of 0.5% to 10%, preferably 1%-2%. Oral formulations include excipients, such as pharmaceutical grades of mannitol, lactose, starch, magnesium stearate, sodium saccharine, cellulose, and magnesium carbonate. These compositions take the form of solutions, suspensions, tablets, pills, capsules, sustained release formulations or powders and contain 10%-95% of active ingredient, preferably 25%-70%.

Topical application can result in transdermal or intradermal delivery. Topical administration can be facilitated by co-administration of the agent with cholera toxin or detoxified derivatives or subunits thereof or other similar bacterial toxins (See Glenn et al., Nature 391, 851 (1998)). Co-administration can be achieved by using the components as a mixture or as linked molecules obtained by chemical crosslinking or expression as a fusion protein.

Alternatively, transdermal delivery can be achieved using a skin patch or using transferosomes (Paul et al., Eur. J. Immunol. 25, 3521-24 (1995); Cevc et al., Biochem. Biophys. Acta 1368, 201-15 (1998)).

### VI. Kits

The invention further provides therapeutic products. The products comprise a glass vial and instructions. The glass vial contains a formulation comprising about 10 mg to about 250 mg of a humanized anti Aβ antibody, about 4% mannitol or about 150 mM NaCl, about 5 mM to about 10 mM histidine, and about 10 mM methionine. The instructions include monitoring the patient for cerebral microhemorrhage by MRI or monitoring the patient for vascular amyloid removal by PET scan.

### EXAMPLES

### Example 1

### Materials and Methods

Study Design. The effects of chronic, passive immunization on established VAβ in the PDAPP mouse were examined in two studies. Study A was designed to compare the efficacy of an N-terminal antibody (3D6, recognizing Aβ1-5) with a mid-region antibody (266, recognizing Aβ16-23) at a single dose. Study B was a 3D6 dose-response study. In both studies, 12-month-old, female, heterozygous PDAPP mice were divided into groups of 40; the groups were matched as closely as possible for age and transgenic parent. In a separate assessment, a group of 40 animals was sacrificed at t₀ determine vascular amyloid levels at 12 months of age. As outlined in Table 1, mice in treatment groups were injected intraperitoneally with murine monoclonal antibodies 3D6 γ2a (at 3 dose levels), 266 γ1, or TY11-15 (as negative control). All treated animals received an initial loading dose of 250% of the planned weekly dose. Doses per animal were calculated based on the historical average weight of a PDAPP mouse in this age range, 50 grams. Animals were treated weekly for approximately 6 months (26 weeks). After termination of the in-life phase, VAβ and microhemorrhage presence and extent were evaluated. All work was performed in accordance with Elan IACUC guidelines.

Preparation of antibodies. Preparation methods for antibodies 3D6 (recognizing Aβ 1-5), 266 (recognizing Aβ 16-23), and 12A11 (recognizing Aβ 3-7) have been described previously (see K. Johnson-Wood et al., Proc Natl Acad Sci USA 94, 1550-5 (1997), P. Seubert et al., Nature 359, 325-7 (1992), F. Bard et al., Proc Natl Acad Sci USA 100, 2023-8 (2003)). TY11/15 (IgG₂ₐ isotype) served as the irrelevant control antibody. It recognizes an unknown human lymphocyte antigen and does not recognize mouse lymphocytes. Antibodies 3D6 and 12A11 were labeled with NHS-biotin as described previously (see P. Seubert et al., Nature 359, 325-7 (1992)).

Brain tissue preparation for histochemistry. Animals were deeply anesthetized with isofluorane and perfused with saline intracardially. One hemisphere from each brain was immersion-fixed for 48 hours in 4% paraformaldehyde at 4° C. and sectioned coronally at 40 μm on a vibrating-blade microtome. The sections were stored in antifreeze solution (30% glycerol/30% ethylene glycol in 40 mM Na₂HPO₄, pH 7.4) at −20° C. prior to immunostaining. Four to 6 sections, spanning the rostral hippocampal level at 240-μm intervals, were selected from each brain for analysis. Brains and sections in which the frontal cortex was damaged during the removal from the skull were excluded from analyses. Final numbers are indicated in the Results. Sections were stained and analyzed by investigators blinded to the treatment status.

VAβ and microhemorrhage co-labeling histochemical procedure: Aβ deposits were labeled with biotinylated antibodies 3D6 (3.0 μg/ml) or 12A11 (3.0 μg/ml) in 1% horse serum in PBS overnight at 4° C. The floating sections were then reacted with an avidin-biotinylated horseradish peroxidase complex and developed using 3,3-diaminobenzidene. Sections were then mounted on slides and co-stained with a Perls iron reaction (see M. M. Racke et al., supra) modified by incubation at 37° C. to intensify hemosiderin reaction product. The presence of hemosiderin is an indication of a past microhemorrhage event.

VAβ analysis: 3D6-immunoreactive blood vessels were assessed in each animal by classifying the animal to one of 2 categories reflecting the amount of VAβ: “none to little VAβ” (≦3 amyloid-positive vessels in any single section per animal) or “moderate VAβ” (>3 amyloid-positive vessels in any single section per animal). This classification method was developed by counting the number of all amyloid-containing vessels in the tissues from studies A and B and using a ROC curve to identify a cutoff that was the optimal one to balance the sensitivity and specificity. Vessels were counted if they contained any amount of amyloid, so both partially cleared vessels and uncleared vessels were counted. Pairwise comparisons using Fisher's Exact Test (FET) were performed to identify significant differences in VAβ. Within each study, the Hochberg method (see Y. Hochberg, Biometrika 75, 800-802 (1988)) was used to adjust for multiple pairwise comparisons.

Microhemorrhage analysis: Each animal was scored on a 0-3 scale for presence, amount, location, and intensity of hemosiderin staining across the sections. A score of “0” indicated very little or no staining, a “1” indicated small punctuate or weak staining in a few sections per animal, a “2” was assigned to contiguous accumulations with greater staining intensity in multiple sections, and “3” reflected the darkest observed staining in most of the sections, usually encompassing most of the surrounding affected vessel. These ratings were designed to reflect the range of hemosiderin-positive staining confined to the present preclinical animal study, and therefore do not represent or translate to ratings of clinical hemorrhagic disorders. Pair-wise comparisons using Fisher's exact test were performed to test for differences between treatment groups. Observations were also made regarding the morphological appearance of the immunolabeled amyloid and its spatial relationship with hemosiderin. Within each study, the Hochberg method (see Y. Hochberg, supra) was used to adjust for multiple pairwise comparisons.

### Results

Vascular Aβ. VAβ was prominent in the leptomeninges and superficial parenchyma of untreated, 18-month-old PDAPP mice as revealed by thioflavin S staining (FIG. 1a) for compact amyloid and by antibody 3D6 (FIG. 1b), which recognizes both compact and diffuse amyloid in the PDAPP mouse and human AD (FIG. 1c). VAβ was largely confined to the meninges and immediately underlying superficial brain layers (FIG. 1d). It was especially predominant in the cortex, particularly in midline vessels of the sagittal sinus, and similar distributions of VAβ were revealed by thioflavin S (FIG. 1) and antibody 3D6 (FIG. 1b) in both mouse and human tissues (FIG. 1c).

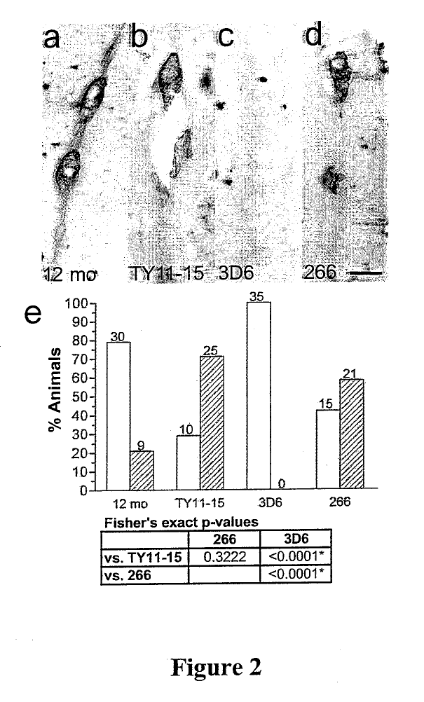

In the single dose comparison study of the N-terminal and mid-region antibodies (Study A), 3D6 at 3 mg/kg completely cleared or prevented VAβ compared with either 266 or TY11-15 (FIG. 2a-e); these differences were statistically significant (p-values<0.0001 for both comparisons). 3D6 treatment also lowered the parenchymal amyloid plaque burden by 98% (p<0.0001), while 266 produced no effect. VAβ was present at moderate levels in 23% of 12-month-old mice before the start of treatment. FET p-values<0.025 are statistically significant using the Hochberg method of multiple comparisons.

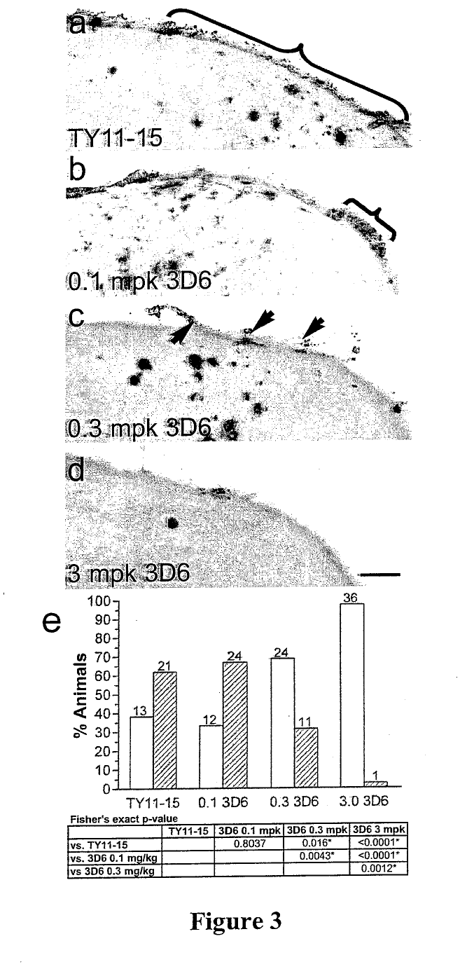

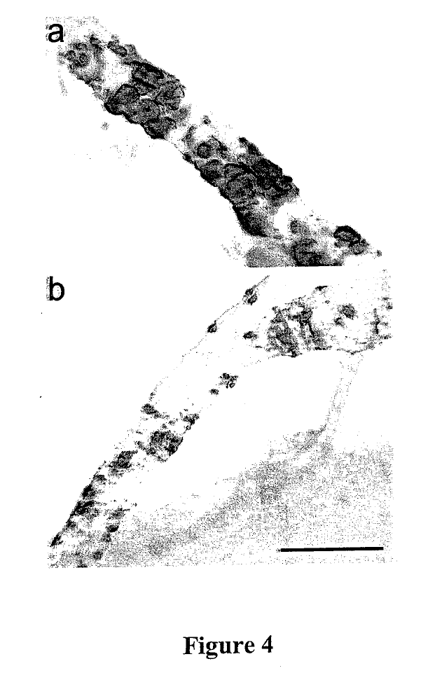

In the 3D6 dose-response study (Study B, FIG. 3a-d), VAβ was again significantly cleared or prevented by 3D6 treatment at the 3.0 mg/kg 3D6 dose level compared to treatment with the TY11-15 control (p<0.001). There was also clearance or prevention of VAβ at the intermediate dose level (0.3 mg/kg) vs. control (p=0.0 16). There was no difference between the 0.1 mg/kg dose group and the TY11-15 control group (p=0.8037). FET p-values<0.025 are statistically significant, using the Hochberg method of multiple comparisons. Although the number of vessels was not significantly different, partial clearance of amyloid was observed from vessels in this group at the microscopic level (FIGS. 4a, b). While intact Aβ forms masses and bands that encompass an unaffected leptomeningeal vessel as shown in FIG. 4a, VAβ has a patchy, eroded appearance during partial clearance (FIG. 4b). This morphology is not seen in untreated mice.

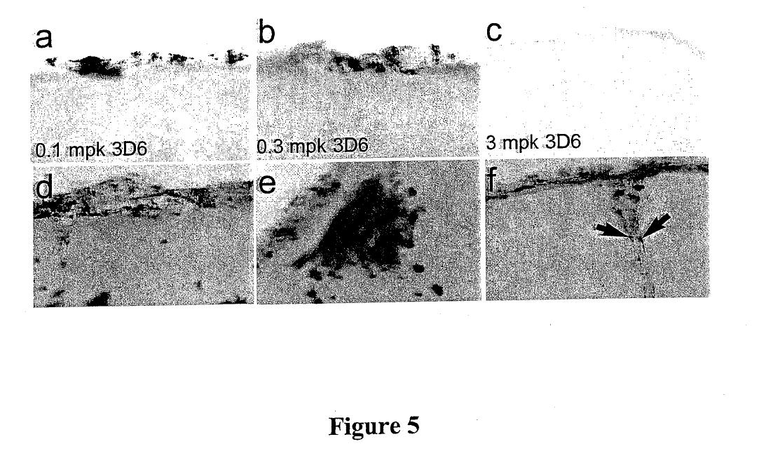

Hemosiderin rating. In order to distinguish the subtle differences among treatment groups, a hemosiderin rating scale was developed that reflects the range of staining densities found within this study. Hemosiderin staining, indicative of microhemorrhage, was limited and confined to the structural boundaries of the vasculature without spread into the surrounding parenchyma. Focal hemosiderin deposits were found in vessels of the leptomeninges of the cortex (FIG. 5a-f) and the hippocampal thalamic interface, the sagittal sinus vessels at the medial cortex, a few parenchymal vessels at right angles and connected to the leptomeningeal vessels. Hemosiderin was usually concentrated within macrophage-like cells in these areas. The foci of hemosiderin were often associated with altered VAβ morphology: instead of the characteristic distinct bands and scales of VAβ deposition (e.g., FIG. 4a), the amyloid had an unusual patchy, degraded appearance (e.g., FIG. 4b) or was completely absent. These features were particularly notable in the leptomeningeal (FIG. 5) and sagittal sinus vessels, which often displayed well-developed VAβ morphology in the TY11-15 control groups and untreated mice (FIGS. 2 and 3).

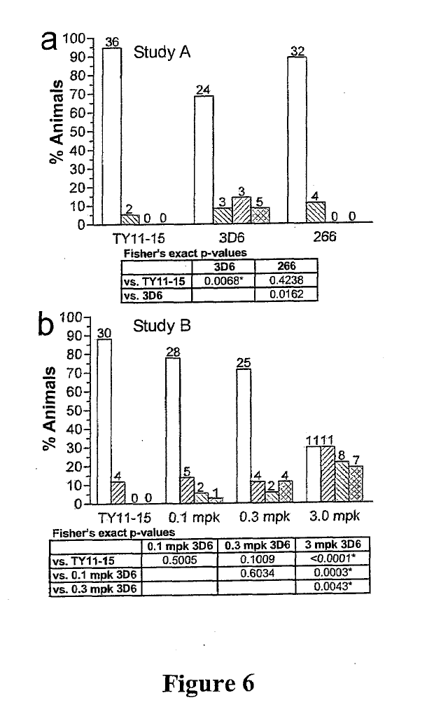

Hemosiderin staining was predominantly absent or mild in all treatment groups, with the majority of animals across groups having scores of 0 or 1 (FIG. 6). Scores in the 3D6 3 mg/kg treatment groups of both studies were significantly higher, indicating that these were more likely than the control to have hemosiderin scores greater than 0. In Study A, the distributions of hemosiderin scores were similar in the TY11-15 and 266 groups indicating that treatment with 266 antibody was not likely to increase hemosiderin scores. In Study B, the incidence of microhemorrhage was shown to be mitigated by dose. No significant differences were found between the TY11-15 control group and the 0.1 mg/kg 3D6 and 0.3 mg/kg 3D6 groups, indicating that the low and intermediate doses were not likely to increase hemosiderin ratings beyond baseline levels. These differed from the 3.0 mg/kg group, which, again, significantly differed from controls. Compared to Study A, the higher hemosiderin scores in the 3.0 mg/kg group could be due to differences in antibody exposure levels over time and slightly higher baseline levels in the cohort as scores were slightly elevated in the control group.

Association of VAβ clearance and microhemorrhage. Significant clearance or prevention of vascular amyloid was observed in the 0.3 mg/kg and 3.0 mg/kg groups in both studies. The majority of animals in these groups had hemosiderin scores of 0 or 1, indicating that most brains with reduced VAP had little or no evidence of microhemorrhage. Several examples of hemosiderin-negative vessels with unusually sparse VAβ and an eroded appearance were found in all of the treatment groups (e.g., FIG. 4b); these may be vessels in which amyloid was being cleared in the absence of microhemorrhage. When hemosiderin staining was seen in 0.1 mg/kg 3D6-treated animals (FIG. 5d), it was typically accompanied a patchy, perivascular distribution of amyloid, possibly indicative of clearance in progress. These perivascular patches of amyloid occurred at sites of vessel-associated hemosiderin labeling within the cortical meninges, parenchyma, and the sagittal sinus vessels. Hemosiderin staining in the 0.3 mg/kg 3D6-treated animals also accompanied a patchy, perivascular distribution of amyloid (FIG. 5e). The amyloid morphology was similar to that in the 0.1 mg/kg 3D6-treated animals, but amyloid was less abundant, and patchy, perivascular distribution of amyloid (FIG. 5e). The amyloid morphology was similar to that in the 0.1 mg/kg 3D6-treated animals, but amyloid was less abundant, and some hemosiderin-positive vessels were cleared of amyloid. Both complete and partial amyloid removal were observed at sites of vessel-associated hemosiderin staining, including the cortical meninges and parenchyma. In contrast, hemosiderin-positive vessels in the 3 mg/kg 3D6-treated animals were often completely devoid of amyloid (FIG. 5f). This feature was never observed in untreated mice and likely illustrates a residual hemosiderin “footprint” that occurred in a subset of vessels with complete VAβ removal. Another feature in these areas was the presence of cells that have phagocytosed hemosiderin (FIG. 5f). These macrophage-like cells were not immunoreactive for Aβ and therefore appear to be a separate population from the microglia and macrophages that remove plaque-associated amyloid.

### Discussion

CAA has been identified as an independent risk factor for cognitive impairment and is associated with significant pathologies such as hemorrhage and ischemic damage (see S. M. Greenberg et al., Stroke 35, 2616-9 (2004)). In typical cases, progressive CAA leads to the destruction of smooth muscle cells in the meningeal and parenchymal vasculature, presumably leading to tonal impairment and compromise of both perfusion and perivascular clearance systems (see R. Christie et al., Am J Pathol 158, 1065-71 (2001), S. D. Preston et al., Neuropathol Appl Neurobiol 29, 106-17 (2003)). We show here for the first time evidence of the near-complete clearance or prevention of VAβ by an N-terminal-specific Aβ antibody (3D6) in a chronic immunotherapeutic treatment paradigm with peripherally administered antibody. Although an understanding of mechanism is not required for practice of the invention, the effect was likely dependent on the ability to robustly bind deposited amyloid, since a mid-region Aβ antibody (266), which binds deposited Aβ in vivo much less avidly, showed no evidence of clearing or preventing VAβ. Although a growing body of evidence suggests that the formation and composition of vascular amyloid may differ from that of parenchymal plaques (see M. C. Herzig et al., Nat Neurosci 7, 954-60 (2004)), antibody 3D6 is competent to clear both forms and thus has a broad spectrum of amyloid-reducing activity.

Previous studies investigating the effects of Aβ immunotherapy and brain microvasculature in APP transgenic mice have reported an increased incidence of microhemorrhage. However, a clear cause-and-effect relationship between VAP and microhemorrhage has not been described. In this report we showed that the majority of deposited vascular amyloid was cleared without inducing microhemorrhage and augmented earlier observations by demonstrating that the incidence of microhemorrhage was associated with VAβ removal. Moreover, the limited areas with microhemorrhages were focally restricted to the architectural boundaries of the vasculature that did not involve the parenchyma. These were associated with either partial or complete removal of VAβ. Importantly, microhemorrhage could be significantly mitigated by modulating the antibody dose within ranges that still effectively cleared parenchymal amyloid plaques.

Racke and colleagues (see M. M. Racke et al., supra) reported the infrequent occurrence of microhemorrhage in PDAPP mice following a significantly larger dose of 3D6 after a 6-week treatment period. Notably, the extent of the reported microhemorrhage was also larger than any observed incidence in the current study, in keeping with our findings of a positive correlation between antibody dose and microhemorrhage scores. Our observations agree with their findings regarding the inability of 266 to bind deposited amyloid or induce microhemorrhage and extend these findings to show that 266 is also not able to clear VAβ.

In APP transgenic mice with very severe VAβ pathology (APP23 mice) cerebral hemorrhage occurs spontaneously and, similarly to human patients, likely is a result of derangement and loss of smooth muscle cells and other destructive consequences of Aβ-related toxicity (see R. Christie et al., supra). Passive immunization of APP23 mice using an N-terminal region Aβ antibody initially exacerbated the incidence and extent of the baseline hemorrhage (see M. Pfeifer et al., Science 298, 1379 (2002)). However, subsequent ultrastructural studies could not find structural differences in the vasculature of treated and non-immunized control (see G. J. Burbach et al., supra). The conclusion was that immunotherapy did not lead to or exacerbate overt damage to the vascular wall, despite the severity of the baseline VAβ pathology. The present study differs from the previous report by examining a model with little spontaneous microhemorrhage and in which Aβ and hemosiderin were co-labeled. We documented the co-localization of Aβ removal and microhemorrhage and found that focal microhemorrhage occurred only in a subset of vessels being cleared of amyloid. Since our quantitation method did not distinguish between partially cleared and intact VAβ, the absolute degree of clearance was likely underestimated.

The relationship between clearance of parenchymal and vascular amyloid is not entirely understood. However, recent reports indicate a co-modulatory relationship likely exists between the two pathologies which may be further clarified in the context of plaque removal (see D. M. Wilcock et al, supra; M. C. Herzig et al., supra; J. A. Nicoll et al., J Neuropathol Exp Neurol 65, 1040-8 (2006)). For example, breeding mutant APP mice with heavy VAβ to those with heavy parenchymal plaque loads actually decreases VAβ, suggesting that plaques can provide a template for Aβ that would otherwise deposit onto the vasculature (see M. C. Herzig et al., supra). Conversely, Wilcock and colleagues (see D. M. Wilcock et al, supra) showed an increase in both VAβ during the course of parenchymal plaque removal in a passive immunization paradigm, suggesting Aβ displacement from parenchymal to vascular compartments may occur during the course of immunotherapy.

In the current study we demonstrate that VAβ can be nearly completely cleared or prevented following passive immunization, which is accompanied by an increased incidence of microhemorrhage that could be diminished by antibody dosage. AD patients and APP transgenic mouse models of AD both show increased incidence of microhemorrhage associated with the progression of VAβ. The microhemorrhage described here could potentially be explained by an increase in VAβ during the period of clearance of parenchymal plaques as described by Wilcock (see D. M. Wilcock et al, supra), which in the present model would be expected to be transient, since VAβ was ultimately cleared by the termination of the study. Alternatively, in the Wilcock study, a different mouse model and antibody was used, thus the VAβ changes may reflect a fundamental mechanistic difference in regards to different antibody epitopes and animal models. In any event, it seems likely that the eventual cumulative incidence of microhemorrhage may actually be lower following 3D6 treatment, assuming that the removal of existing VAβ and prevention of further deposition will have a prophylactic effect towards further microhemorrhage associated with progressive VAβ. In other words, both treatment-related VAβ and VAβ-contingent microhemorrhage might be transient phenomena that would not persist after VAβ is ultimately removed. Taken together, findings from preclinical models indicate that mechanisms associated with the formation and clearance of VAβ warrant further study. Importantly, a recent study has shown that immunization with full length Aβ peptide in TG 2576 mice actually improves the integrity of the blood brain barrier (i.e. reduced the permeability of Evan's blue), suggesting that these multiple factors might in fact have a positive impact on the vasculature (see D. L. Dickstein et al., supra). It should be noted the immunization with the total Aβ peptide results in antibodies directed primarily to the N terminus similar to the epitope of 3D6.

About 80% of AD patients are affected by at least mild CAA, with clinically detrimental consequences of hemorrhage, white matter degeneration, ischemia and inflammation (see S. M. Greenberg et al., supra). The findings from our study provide evidence that Aβ immunotherapy can potentially reverse or prevent the progression of a significant vascular pathology for which there is currently no treatment and further extend the potential therapeutic benefits of anti-Aβ immunotherapy.

### Example 2

### Materials and Methods

The effects of structural changes induced by amyloid on smooth muscle cells (SMC) and extracellular matrix (ECM) of PDAPP mouse vessels and the effects of passive immunization on SMC and ECM of PDAPP mouse vessels were examined.

Mice were immunized weekly for either 3 or 9 months with 1 or 3 mg/Kg of 3D6 antibody. High-resolution, quantitative immunohistochemical (IHC) analyses of vascular components (α-actin for SMC and collagen-IV for ECM) were performed on meningeal vessels from the sagittal sinus, where VAβ deposition is prominent (˜70% of vessels affected). Microhemorrhage events were monitored by hemosiderin detection or ferritin immunohistochemistry.

### Results

In the current study we demonstrate that changes in the vascular wall are invariably associated with VAβ, and they included both degeneration (decreased thickness) and hyperplasia/hypertrophy (increased thickness) of SMC and ECM. These two contrasting findings were often observed in the same vessel and were not present in wild type animals or PDAPP vessels lacking amyloid. The extreme degrees of thickening and thinning of the SM resulted in a widely variable vascular phenotype in untreated PDAPP mice.

Passive immunotherapy restored the pattern of vascular SMC and ECM thicknesses and reduced the phenotypic variability in a dose- and time-dependent manner, with the high dose of 3D6 reaching control levels (wild type) at 9 months (p>0.05). Although the incidence of microhemorrhage increased in the 3-month group, it reduced to control levels after 9 months of treatment (p>0.05). Our results suggest that passive immunotherapy allows the recovery of meningeal vessels from amyloid-induced structural changes. Furthermore, the treatment-related increase in microhemorrhage appears to be a transient event that resolves during VAβ clearance. Mechanisms of repair may be triggered by VAβ removal, which ultimately lead to recovery from vascular dysfunction.

Although the foregoing invention has been described in detail for purposes of clarity of understanding, it will be obvious that certain modifications may be practiced within the scope of the appended claims. All publications and patent documents cited herein, as well as text appearing in the figures, are hereby incorporated by reference in their entirety for all purposes to the same extent as if each were so individually denoted.

What is claimed is:

1. A method of treating CAA, comprising
administering to a patient having or suspected of having CAA an effective regime of an agent, wherein the agent is antibody that is specific for the N-terminus of Aβ or induces such an antibody after administration to the patient and thereby treating the patient.

2. The method of claim 1, wherein the agent is an antibody.

3. The method of claim 2, wherein the agent is an antibody that binds within residues 1-5 of Aβ.

4. The method of claim 2, wherein the antibody is a humanized, human, or chimeric antibody.

5. The method of claim 4, wherein the antibody is humanized 3D6 or humanized 12A11.

6. The method of claim 5, wherein the 3D6 humanized antibody is bapineuzumab.

7. The method of claim 1, wherein the agent is a fragment of Aβ.

8. The method of claim 7, wherein the fragment begins at residue 1 of Aβ and ends at one of residues 5-10 of Aβ.

9. The method of claim 7, wherein the fragment is Aβ 1-7.

10. The method of claim 7, wherein the fragment of Aβ is administered with a pharmaceutically acceptable adjuvant.

11. The method of claim 7, wherein the fragment of Aβ is linked to a carrier that helps the fragment induce antibodies to the fragment.

12. The method of claim 11, wherein the carrier is linked to the C-terminus of the fragment.

13. The method of claim 1, further comprising determining that a patient has CAA, wherein the determining step occurs before the administration step.

14. The method of claim 13, wherein the determining step determines that a patient is suffering from a clinical symptom of CAA.

15. The method of claim 1, wherein the patient lacks plaques characteristic of Alzheimer's disease in the brain.

16. The method of claim 14, wherein the patient lacks symptoms of Alzheimer's disease.

17. The method of claim 1, wherein the patient has had a heart attack or stroke.

18. The method of claim 1, wherein the dosage of the antibody is between about 0.01 to about 5 mg/kg.

19. The method of claim 1, wherein the dosage of the antibody is between about 0.1 to about 5 mg/kg.

20. The method of claim 18, wherein the dosage is about 0.5 mg/kg.

21. The method of claim 18, wherein the dosage is about 1.5 mg/kg.

22. The method of claim 18, wherein the dosage is between about 0.5 to about 3 mg/kg.

23. The method of claim 18, wherein the dosage is between about 0.5 to about 1.5 mg/kg.

24. The method of claim 18, wherein the dosage is administered on multiple occasions.

25. The method of claim 18, wherein the dosage is administered is weekly to quarterly.

26. The method of claim 18, wherein the dosage is administered every 13 weeks.

27. The method of claim 1, wherein the antibody is administered intravenously or subcutaneously.

28. The method of claim 1, further comprising monitoring for changes in signs or symptoms of CAA responsive to the administrating step.

29. The method of claim 1, further comprising administering a second agent effective to treat CAA.

30. A method of effecting prophylaxis against CAA, comprising administering to a patient susceptible to CAA an effective regime of an agent, wherein the agent is an antibody that is specific for the N-terminus of Aβ or induces such an antibody after administration to the patient and thereby effecting prophylaxis of the patient.

31. Use of an agent, wherein the agent is an antibody that is specific for the N-terminus of Aβ or induces such an antibody after administration to the patient, in the treatment or prophylaxis of Alzheimer's disease.

32. A method of reducing vascular amyloid in a patient, comprising administering an antibody that is specific for the N-terminus of Aβ in a treatment regime associated with efficacious vascular amyloid removal and reduced incidence of cerebral microhemorrhage.

33. The method of claim 32, further comprising monitoring the patient for cerebral microhemorrhage by MRI.

34. The method of claim 32, further comprising
monitoring the patient for vascular amyloid removal by PET scan.

35. The method of claim 32, wherein the treatment regime is a chronic treatment regime.

36. The method of claim 32, wherein the treatment regime comprises an antibody dosage between 0.01 and 5 mg/kg body weight of the patient and administered weekly to quarterly.

37. The method of claim 36, wherein the dosage of the antibody is 0.1 to 5 mg/kg.

38. The method of claim 36, wherein the dosage is about 0.5 mg/kg.

39. The method of claim 36, wherein the dosage is about 1.5 mg/kg.

40. The method of claim 36, wherein the dosage is between about 0.5 to about 3 mg/kg.

41. The method of claim 36, wherein the dosage is between about 0.5 to about 1.5 mg/kg.

42. The method of claim 36, wherein the dosage is administered every 13 weeks.

43. The method of claim 32, wherein the antibody is administered intravenously or subcutaneously.

44. The method of claim 32, wherein the antibody binds within residues 1-5 of Aβ.

45. The method of claim 32, wherein the antibody is a humanized, human, or chimeric antibody.

46. The method of claim 45, wherein the antibody is humanized 3D6 or humanized 12A 11.

47. The method of claim 46, wherein the humanized antibody is bapineuzumab.

48. A method of treating Alzheimer's disease, comprising administering an antibody that is specific for the N-terminus of Aβ at a dose that reduces or inhibits development of vascular amyloidogenic pathology, minimizes microhemorrhage, and or reduces or inhibits development of Aβ plaques.

49. The method of claim 48, wherein the antibody binds within residues 1-5 of Aβ.

50. The method of claim 48, wherein the antibody is a humanized, human, or chimeric antibody.

51. The method of claim 50, wherein the antibody is humanized 3D6 or humanized 12A 11.

52. The method of claim 51, wherein the humanized antibody is bapineuzumab.

53. A method of treating Alzheimer's disease, comprising administering an antibody that is specific for the N-terminus of Aβ at a dose that reduces or inhibits development of vascular amyloidogenic pathology, minimizes microhemorrhage, and or reduces or inhibits development of neuritic pathology.

54. The method of claim 53, wherein the antibody binds within residues 1-5 of Aβ.

55. The method of claim 53, wherein the antibody is a humanized, human, or chimeric antibody.

56. The method of claim 55, wherein the antibody is humanized 3D6 or humanized 12A11.

57. The method of claim 56, wherein the humanized antibody is bapineuzumab.

58. A method of treating Alzheimer's disease, comprising administering an antibody that is specific for the N-terminus of Aβ at a dose that reduces or inhibits vascular amyloidogenic pathology, minimizes microhemorrhage, and or improves patient's cognitive function.

59. The method of claim 58, wherein the antibody binds within residues 1-5 of Aβ.

60. The method of claim 58, wherein the antibody is a humanized, human, or chimeric antibody.

61. The method of claim 60, wherein the antibody is humanized 3D6 or humanized 12A11.

62. The method of claim 61, wherein the humanized antibody is bapineuzumab.

63. The method of any of claims 48-62, wherein the reduction or inhibition of vascular amyloidogenic pathology is a prevention of accumulation of vascular Aβ or clearance of vascular AD.

64. A kit for treatment of CAA, comprising:
a. a glass vial containing a formulation; and
b. instructions to monitor a patient to whom the formulation is administered for CAA.

65. A kit for treatment of CAA, comprising:
a. a glass vial containing a formulation comprising about 0.5 to 3 mg/kg of a humanized anti-Aβ antibody; and
b. instructions to monitor a patient to whom the formulation is administered for CAA comprising:
i. monitoring the patient for cerebral microhemorrhage by MRI, or
ii. monitoring the patient for vascular amyloid removal by PET scan.

66. A kit for treatment of CAA, comprising:
a. a glass vial containing a formulation comprising:
i. between about 10 mg to about 250 mg of a humanized anti-Aβ antibody,
ii. about 4% mannitol or about 150 mM NaCl,
iii. about 5 mM to about 10 mM histidine, and
iv. about 10 mM methionine; and
b. instructions to monitor a patient to whom the formulation is administered for CAA comprising:
i. monitoring the patient for cerebral microhemorrhage by MRI, or
ii. monitoring the patient for vascular amyloid removal by PET scan.

67. A kit for treatment of Alzheimer's disease, comprising:
a. a glass vial containing a formulation comprising:
i. between about 10 mg to about 250 mg of a humanized anti-Aβ antibody,
ii. about 4% mannitol or about 150 mM NaCl,
iii. about 5 mM to about 10 mM histidine, and
iv. about 10 mM methionine; and
b. instructions to monitor a patient to whom the formulation is administered for Alzheimer's disease comprising:
i. monitoring the patient for cerebral microhemorrhage by MRI, or
ii. monitoring the patient for vascular amyloid removal by PET scan.

68. A kit for treatment of CAA and Alzheimer's disease, comprising:
a. a glass vial containing a formulation comprising:
i. between about 10 mg to about 250 mg of a humanized anti-Aβ antibody,
ii. about 4% mannitol or about 150 mM NaCl,
iii. about 5 mM to about 10 mM histidine, and
iv. about 10 mM methionine; and
b. instructions to monitor a patient to whom the formulation is administered for CAA and Alzheimer's disease comprising:
i. monitoring the patient for cerebral microhemorrhage by MRI, or
ii. monitoring the patient for vascular amyloid removal by PET scan.

69. The method of claim 2, wherein the antibody is administered in a regime sufficient to maintain an average serum concentration of the antibody in the patient in a range of 1-15 μg antibody/ml serum and thereby treating the patient.

70. The method of claim 69, wherein the average serum concentration is within a range of 1-10 μg antibody/ml serum.

71. The method of claim 70, wherein the average serum concentration is within a range of 1-5 μg antibody/ml serum.

72. The method of claim 71, wherein the average serum concentration is within a range of 2-4 μg antibody/ml serum.

73. The method of claim 69, wherein the antibody is administered intravenously.

74. The method of claim 73, wherein a dose of 0.5-1.0 mg/kg is administered monthly.

75. The method of claim 74, wherein a dose of 0.1-1.0 mg/kg is administered monthly.

76. The method of claim 2, wherein the antibody is administered subcutaneously.

77. The method of claim 76, wherein the antibody is administered at a frequency between weekly and monthly.

78. The method of claim 76, wherein the antibody is administered weekly or biweekly.

79. The method of claim 76, wherein the antibody is administered at a dose of between about 0.01 to about 0.35 mg/kg.

80. The method of claim 76, wherein the antibody is administered at a dose of between about 0.05 to about 0.25 mg/kg.

81. The method of claim 76, wherein the antibody is administered at a dose of between about 0.015 to about 0.2 mg/kg weekly to biweekly.

82. The method of claim 76, wherein the antibody is administered at a dose of between about 0.05 to about 0.15 mg/kg weekly to biweekly.

83. The method of claim 76, wherein the antibody is administered at a dose of between about 0.05 to about 0.07 mg/kg weekly.

84. The method of claim 76, wherein the antibody is administered at a dose of 0.06 mg/kg weekly.

85. The method of claim 76, wherein the antibody is administered at a dose of between about 0.1 to about 0.15 mg/kg biweekly.

86. The method of claim 2, wherein the average serum concentration of the antibody is maintained for at least six months.

87. The method of claim 2, wherein the average serum concentration of the antibody is maintained for at least one year.

88. The method of claim 2, further comprising measuring the concentration of antibody in the serum and adjusting the regime if the measured concentration falls outside the range.

89. The method of claim 2, wherein the antibody is administered subcutaneously at a dose of between about 0.01 to about 0.6 mg/kg and a frequency of between weekly and monthly.

90. The method of claim 2, wherein the antibody is administered at a dose of between about 0.05 to about 0.5 mg/kg.

91. The method of claim 89, wherein the antibody is administered at a dose of between about 0.05 to about 0.25 mg/kg.

92. The method of claim 89, wherein the antibody is administered at a dose of between about 0.015 to about 0.2 mg/kg weekly to biweekly.

93. The method of claim 89, wherein the antibody is administered at a dose of between about 0.05 to about 0.15 mg/kg weekly to biweekly.

94. The method of claim 89, wherein the antibody is administered at a dose of between about 0.05 and about 0.07 mg/kg weekly.

95. The method of claim 89, wherein the antibody is administered at a dose of 0.06 mg/kg weekly.

96. The method of claim 89, wherein the antibody is administered at a dose of between about 0.1 to about 0.15 mg/kg biweekly.

97. The method of claim 89, wherein the antibody is administered at a dose of between about 0.1 to about 0.3 mg/kg monthly.

98. The method of claim 89, wherein the antibody is administered at a dose of 0.2 mg/kg monthly.

99. The method of claim 2, wherein the antibody is administered at a dose of between about 1 to about 40 mg and a frequency of between weekly and monthly.

100. The method of claim 99, wherein the antibody is administered at a dose of between about 5 to about 25 mg.

101. The method of claim 99, wherein the antibody is administered at a dose of between about 2.5 to about 15 mg.

102. The method of claim 99, wherein the antibody is administered at a dose of between about 1 to about 12 mg weekly to biweekly.

103. The method of claim 99, wherein the antibody is administered at a dose of between about 2.5 to about 10 mg weekly to biweekly.

104. The method of claim 99, wherein the antibody is administered at a dose of between about 2.5 to about 5 mg weekly.

105. The method of claim 99, wherein the antibody is administered at a dose of between about 4 to about 5 mg weekly.

106. The method of claim 98, wherein the antibody is administered at a dose of between about 7 to about 10 mg biweekly.


SEQUENCE LISTING (53 sequences)

SEQ ID NO: 1 - protein, 112 aa, Artificial [sequence omitted]

SEQ ID NO: 2 - protein, 119 aa, Artificial [sequence omitted]

SEQ ID NO: 3 - protein, 112 aa, Artificial
  Synthetic Humanized 3D6 Light Chain Variable Region (version 2)
YVVMTQSPLSLPVTPGEPASISCKSSQSLLDSDGKTYLNWLLQKPGQSPQRLIYLVSKLDSGVPDRFSGSGSGTDFTLKISRVEAEDVGVYYCWQGTHFPRTFGQGTKVEIK

SEQ ID NO: 4 - protein, 119 aa, Artificial
  Synthetic Humanized 3D6 Heavy Chain Variable Region (version 2)
EVQLLESGGGLVQPGGSLRLSCAASGFTFSNYGMSWVRQAPGKGLEWVASIRSGGGRTYYSDNVKGRFTISRDNAKNSLYLQMNSLRAEDTALYYCVRYDHYSGSSDYWGQGTLVTVSS

SEQ ID NO: 5 - protein, 219 aa, Artificial
  Synthetic Humanized 3D6 Light Chain
DVVMTQSPLSLPVTLGQPASISCKSSQSLLDSDGKTYLNWLQQRPGQSPRRLIYLVSKLDSGVPDRFSGSGSGTDFTLKISRVEAEDVGVYYCWQGTHFPRTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 6 - protein, 449 aa, Artificial
  Synthetic Humanized 3D6 Heavy Chain
QVQLVQSGGGLVQPGGSLRLSCAGSGFTFSNYGMSWVRQAPGKGLEWVASIRSGGGRTYYSDNVKGRFTISRENAKNSLYLQMNSLRAEDTAVYYCVRYDHYSGSSDYWGQGTLVTVSSASTKGPSVFPLAPSSKSTSGGTAALGCLVKDYFPQPVTVSWNSGALTSGVHTFPAVLQSSGLYSLSSVVTVPSSSLGTQTYICNVNHKPSNTKVDKKVEPKSCDKTHTCPPCPAPQLLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - protein, 112 aa, Artificial
  Synthetic Humanized 12A11 Light Chain
DVVMTQSPLSLPVTPGEPASISCRSSQSIVHSNGNTYLEWYLQKPGQSPQLLIYKVSNRFSGVPDRFSGSGSGTDFTLKISRVEAEDVGVYYCFQSSHVPLTFGQGTKLEIK

SEQ ID NO: 8 - protein, 120 aa, Artificial
  Synthetic Humanized 12A11 Heavy Chain (version 1)
QVQLVESGGGVVQPGRSLRLSCAFSGFSLSTSGMSVGWIRQAPGKGLEWLAHIWWDDDKYYNPSLKSRLTISKDTSKNTVYLQMNSLRAEDTAVYYCARRTTTADYFAYWGQGTTVTVSS

SEQ ID NO: 9 - protein, 120 aa, Artificial
  Synthetic Humanized 12A11 Heavy Chain (version 2)
QVQLVESGGGVVQPGRSLRLSCAFSGFTLSTSGMSVGWIRQAPGKGLEWVAHIWWDDDKYYNPSLKSRFTISKDTSKNTLYLQMNSLRAEDTAVYYCARRTTTADYFAYWGQGTTVTVSS

SEQ ID NO: 10 - protein, 120 aa, Artificial
  Synthetic Humanized 12A11 Heavy Chain (version 2.1)
QVQLVESGGGVVQPGRSLRLSCAFSGFTLSTSGMSVGWIRQAPGKGLEWVAHIWWDDDKYYNPSLKSRFTISKDNSKNTLYLQMNSLRAEDTAVYYCARRTTTADYFAYWGQGTTVTVSS

SEQ ID NO: 11 - protein, 120 aa, Artificial
  Synthetic Humanized 12A11 Heavy Chain (version 3)
QVQLVESGGGVVQPGRSLRLSCAFSGFTLSTSGMSVGWIRQAPGKGLEWVAHIWWDDDKYYNPSLKSRFTISRDNSKNTLYLQMNSLRAEDTAVYYCARRTTTADYFAYWGQGTTVTVSS

SEQ ID NO: 12 - protein, 120 aa, Artificial
  Synthetic Humanized 12A11 Heavy Chain (version 4.1)
QVQLVESGGGVVQPGRSLRLSCAFSGFTLSTSGMSVGWIRQAPGKGLEWLAHIWWDDDKYYNPSLKSRLTISKDTSKNTVYLQMNSLRAEDTAVYYCARRTTTADYFAYWGQGTTVTVSS

SEQ ID NO: 13 - protein, 120 aa, Artificial
  Synthetic Humanized 12A11 Heavy Chain (version 4.2)
QVQLVESGGGVVQPGRSLRLSCAFSGFSLSTSGMSVGWIRQAPGKGLEWVAHIWWDDDKYYNPSLKSRLTISKDTSKNTVYLQMNSLRAEDTAVYYCARRTTTADYFAYWGQGTTVTVSS

SEQ ID NO: 14 - protein, 120 aa, Artificial
  Synthetic Humanized 12A11 Heavy Chain (version 4.3)
QVQLVESGGGVVQPGRSLRLSCAFSGFSLSTSGMSVGWIRQAPGKGLEWLAHIWWDDDKYYNPSLKSRFTISKDTSKNTVYLQMNSLRAEDTAVYYCARRTTTADYFAYWGQGTTVTVSS

SEQ ID NO: 15 - protein, 120 aa, Artificial
  Synthetic Humanized 12A11 Heavy Chain (version 4.4)
QVQLVESGGGVVQPGRSLRLSCAFSGFSLSTSGMSVGWIRQAPGKGLEWLAHIWWDDDKYYNPSLKSRLTISKDTSKNTLYLQMNSLRAEDTAVYYCARRTTTADYFAYWGQGTTVTVSS

SEQ ID NO: 16 - protein, 120 aa, Artificial
  Synthetic Humanized 12A11 Heavy Chain (version 5.1)
QVQLVESGGGVVQPGRSLRLSCAFSGFTLSTSGMSVGWIRQAPGKGLEWVAHIWWDDDKYYNPSLKSRLTISKDTSKNTVYLQMNSLRAEDTAVYYCARRTTTADYFAYWGQGTTVTVSS

SEQ ID NO: 17 - protein, 120 aa, Artificial
  Synthetic Humanized 12A11 Heavy Chain (version 5.2)
QVQLVESGGGVVQPGRSLRLSCAFSGFTLSTSGMSVGWIRQAPGKGLEWLAHIWWDDDKYYNPSLKSRFTISKDTSKNTVYLQMNSLRAEDTAVYYCARRTTTADYFAYWGQGTTVTVSS

SEQ ID NO: 18 - protein, 121 aa, Artificial
  Synthetic Humanized 12A11 Heavy Chain (version 5.3)
QVQLVESGGGVVQPGRSLRLSCAFSGFTLSTSGMSVGWIRQAPGKGLEWLAHIWWDDDKYYNPSLKSRLTISKDTSKNTLYLQMNSLRAEDTAVYYCARRTTTADYFAYWGQGTTVTVSSV

SEQ ID NO: 19 - protein, 121 aa, Artificial
  Synthetic Humanized 12A11 Heavy Chain (version 5.4)
QVQLVESGGGVVQPGRSLRLSCAFSGFSLSTSGMSVGWIRQAPGKGLEWVAHIWWDDDKYYNPSLKSRFTISKDTSKNTVYLQMNSLRAEDTAVYYCARRTTTADYFAYWGQGTTVTVSSV

SEQ ID NO: 20 - protein, 120 aa, Artificial
  Synthetic Humanized 12A11 Heavy Chain (version 5.5)
QVQLVESGGGVVQPGRSLRLSCAFSGFSLSTSGMSVGWIRQAPGKGLEWVAHIWWDDDKYYNPSLKSRLTISKDTSKNTLYLQMNSLRAEDTAVYYCARRTTTADYFAYWGQGTTVTVSS

SEQ ID NO: 21 - protein, 120 aa, Artificial
  Synthetic Humanized 12A11 Heavy Chain (version 5.6)
QVQLVESGGGVVQPGRSLRLSCAFSGFSLSTSGMSVGWIRQAPGKGLEWLAHIWWDDDKYYNPSLKSRFTISKDTSKNTLYLQMNSLRAEDTAVYYCARRTTTADYFAYWGQGTTVTVSS

SEQ ID NO: 22 - protein, 120 aa, Artificial
  Synthetic Humanized 12A11 Heavy Chain (version 6.1)
QVQLVESGGGVVQPGRSLRLSCAFSGFTLSTSGMSVGWIRQAPGKGLEWVAHIWWDDDKYYNPSLKSRFTISKDTSKNTVYLQMNSLRAEDTAVYYCARRTTTADYFAYWGQGTTVTVSS

SEQ ID NO: 23 - protein, 120 aa, Artificial
  Synthetic Humanized 12A11 Heavy Chain (version 6.2)
QVQLVESGGGVVQPGRSLRLSCAFSGFTLSTSGMSVGWIRQAPGKGLEWVAHIWWDDDKYYNPSLKSRLTISKDTSKNTLYLQMNSLRAEDTAVYYCARRTTTADYFAYWGQGTTVTVSS

SEQ ID NO: 24 - protein, 120 aa, Artificial
  Synthetic Humanized 12A11 Heavy Chain (version 6.3)
QVQLVESGGGVVQPGRSLRLSCAFSGFTLSTSGMSVGWIRQAPGKGLEWLAHIWWDDDKYYNPSLKSRFTISKDTSKNTLYLQMNSLRAEDTAVYYCARRTTTADYFAYWGQGTTVTVSS

SEQ ID NO: 25 - protein, 120 aa, Artificial
  Synthetic Humanized 12A11 Heavy Chain (version 6.4)
QVQLVESGGGVVQPGRSLRLSCAFSGFSLSTSGMSVGWIRQAPGKGLEWVAHIWWDDDKYYNPSLKSRFTISKDTSKNTLYLQMNSLRAEDTAVYYCARRTTTADYFAYWGQGTTVTVSS

SEQ ID NO: 26 - protein, 120 aa, Artificial
  Synthetic Humanized 12A11 Heavy Chain (version 7)
QVQLVESGGGVVQPGRSLRLSCAFSGFTLSTSGMSVGWVRQAPGKGLEWLAHIWWDDDKYYNPSLKSRLTISKDTSKNTVYLQMNSLRAEDTAVYYCARRTTTADYFAYWGQGTTVTVSS

SEQ ID NO: 27 - protein, 120 aa, Artificial
  Synthetic Humanized 12A11 Heavy Chain (version 8)
QVQLVESGGGVVQPGRSLRLSCAFSGFSLSTSGMSVGWIRQAPGKGLEWLAHIWWDDDKYYNPSLKSRLTISKDNSKNTVYLQMNSLRAEDTAVYYCARRTTTADYFAYWGQGTTVTVSS

SEQ ID NO: 28 - protein, 43 aa, Homo sapiens
DAEFRHDSGYEVHHQKLVFFAEDVGSNKGAIIGLMVGGVVIAT

SEQ ID NO: 29 - protein, 16 aa, Plasmodium sp.
EKKIAKMEKASSVFNV

SEQ ID NO: 30 - protein, 10 aa, Hepatitis B virus
FFLLTRILTI

SEQ ID NO: 31 - protein, 19 aa, Homo sapiens
DQSIGDLIAEAMDKVGNEG

SEQ ID NO: 32 - protein, 14 aa, Mycobacterium bovis
QVHFQPLPPAVVKL

SEQ ID NO: 33 - protein, 15 aa, Clostridium tetani
QYIKANSKFIGITEL

SEQ ID NO: 34 - protein, 21 aa, Clostridium tetani
FNNFTVSFWLRVPKVSASHLE

SEQ ID NO: 35 - protein, 16 aa, Human immunodeficiency virus
KQIINMWQEVGKAMYA

SEQ ID NO: 36 - protein, 22 aa, Artificial
  Synthetic fusion protein (AB1-7 - Tetanus toxoid 830-844)
DAEFRHDQYIKANSKFIGITEL

SEQ ID NO: 37 - protein, 28 aa, Artificial
  Synthetic fusion protein (AB1-7 - Tetanus toxoid 947-967)
DAEFRHDFNNFTVSFWLRVPKVSASHLE

SEQ ID NO: 38 - protein, 43 aa, Artificial
  Synthetic fusion protein (AB1-7 - Tetanus toxoid 830-844 + 947-967)
DAEFRHDQYIKANSKFIGITELFNNFTVSFWLRVPKVSASHLE

SEQ ID NO: 39 - protein, 19 aa, Artificial
  Synthetic fusion protein (PADRE - AB1-7)
AKXVAAWTLAAADAEFRHD

SEQ ID NO: 40 - protein, 34 aa, Artificial
  Synthetic fusion protein (3PADRE - AB1-7)
DAEFRHDDAEFRHDDAEFRHDAKXVAAWTLKAAA

SEQ ID NO: 41 - protein, 34 aa, Artificial
  Synthetic fusion protein
AKXVAAWTLKAAADAEFRHDDAEFRHDDAEFRHD

SEQ ID NO: 42 - protein, 20 aa, Artificial
  Synthetic fusion protein
DAEFRHDAKXVAAWTLKAAA

SEQ ID NO: 43 - protein, 24 aa, Artificial
  Synthetic fusion protein
DAEFRHDISQAVHAAHAEINEAGR

SEQ ID NO: 44 - protein, 24 aa, Artificial
  Synthetic fusion protein
FRHDSGYISQAVHAAHAEINEAGR

SEQ ID NO: 45 - protein, 24 aa, Artificial
  Synthetic fusion protein
EFRHDSGISQAVHAAHAEINEAGR

SEQ ID NO: 46 - protein, 34 aa, Artificial
  Synthetic fusion protein
PKYVKQNTLKLATDAEFRHDDAEFRHDDAEFRHD

SEQ ID NO: 47 - protein, 27 aa, Artificial
  Synthetic fusion protein
DAEFRHDPKYVKQNTLKLATDAEFRHD

SEQ ID NO: 48 - protein, 34 aa, Artificial
  Synthetic fusion protein
DAEFRHDDAEFRHDDAEFRHDPKYVKQNTLKLAT

SEQ ID NO: 49 - protein, 27 aa, Artificial
  Synthetic fusion protein
DAEFRHDDAEFRHDPKYVKQNTLKLAT

SEQ ID NO: 50 - protein, 79 aa, Artificial
  Synthetic fusion protein
DAEFRHDPKYVKQNTLKLATEKKIAKMEKASSVFNVQYIKANSKFIGITELFNNFTVSFWLRVPKVSASHLEDAEFRHD

SEQ ID NO: 51 - protein, 56 aa, Artificial
  Synthetic fusion protein
DAEFRHDDAEFRHDDAEFRHDQYIKANSKFIGITELNNFTVSFWLRVPKVSASHLE

SEQ ID NO: 52 - protein, 44 aa, Artificial
  Synthetic fusion protein
DAEFRHDQYIKANSKFIGITELCFNNFTVSFWLRVPKVSASHLE

SEQ ID NO: 53 - protein, 51 aa, Artificial
  Synthetic fusion protein
DAEFRHDQYIKANSKFIGITELCFNNFTVSFWLRVPKVSASHLEDAEFRHD